(19) United States
(12) Patent Application Publication
(10) Pub. No.: US 2008/0139532 A1
(43) Pub. Date: Jun. 12, 2008

(54) Tetrahydrobenzazepine Derivatives as Modulators of Dopamine D3 Receptors (Antipsychotic Agents)

(75) Inventors: Luca Arista, Verona (IT); Giorgio Bonanomi, Verona (IT); Federica Damiani, Verona (IT); Dieter Hamprecht, Verona (IT); Fabrizio Micheli, Verona (IT); Luca Tarsi, Verona (IT); Giovanna Tedesco, Verona (IT)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited, Greenford Middlesex (GB)

(21) Appl. No.: 10/591,782

(22) PCT Filed: Mar. 4, 2005
(86) PCT No.: PCT/EP05/02635

§ 371 Date: Nov. 21, 2007

(30) Foreign Application Priority Data
Mar. 8, 2004 (GB) 0405198.3
Jun. 24, 2004 (GB) 0414204.8

(51) Int. Cl. C07D 403/12; A61K 31/55; A61P 25/30

(52) U.S. Cl. 514/217.01; 540/594

(57) ABSTRACT

The present invention relates to novel compounds of formula (I) or a pharmaceutically acceptable salt thereof, processes for their preparation, intermediates used in these processes, pharmaceutical compositions containing them and their use in therapy, as modulators of dopamine D₃ receptors, e.g. as agents to treat various aspects drug dependency or as antipsychotic agents.

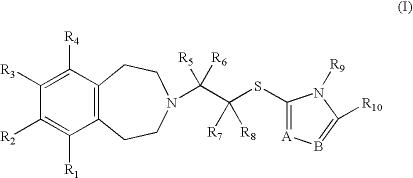

The present invention relates to novel compounds, processes for their preparation, intermediates used in these processes, pharmaceutical compositions containing them and their use in therapy, as modulators of dopamine D₃ receptors, e.g. as agents to treat various aspects of drug dependency or as antipsychotic agents.

WO 2002/40471 (SmithKline Beecham) discloses certain benzodiazepine compounds having activity at the dopamine D₃ receptor.

A new class of compounds which have affinity for dopamine receptors, in particular the dopamine D₃ receptor, has been found. These compounds have potential in the treatment of conditions wherein modulation, especially antagonism/inhibition, of the D₃ receptor is beneficial, e.g. as antipsychotic agents or to treat drug dependency.

The present invention provides a compound of formula (I) or a salt thereof:

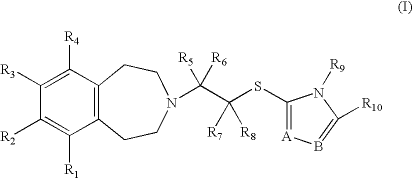

wherein
R₁ and R₄ are independently selected from the group consisting of hydrogen, fluoro, chloro, bromo, C₁₋₂alkyl, C₁alkoxy, haloC₁₋₂alkyl, haloC₁alkoxy, hydroxy, cyano and nitro;
R₂ and R₃ are independently selected from the group consisting of:
   halogen, hydroxy, cyano, nitro, C₁₋₄alkyl, haloC₁₋₄alkyl, C₃₋₆cycloalkyl, C₁₋₄alkoxy, haloC₁₋₄alkoxy, C₁₋₄alkoxyC₁₋₄alkoxy, C₁₋₄alkylthio, C₁₋₄alkoxyC₁₋₄alkyl, C₃₋₆cycloalkylC₁₋₄alkoxy, C₁₋₄alkanoyl, C₁₋₄alkoxycarbonyl, C₁₋₄alkoxycarbonylC₁₋₄alkyl, C₁₋₄alkylsulfonyl, C₁₋₄alkylsulfonyloxy, haloC₁₋₄alkylsulfonyl, haloC₁₋₄alkylsulfonyloxy, C₁₋₄alkylsulfonylC₁₋₄alkyl, C₁₋₄alkylsulfonamido, C₁₋₄alkylsulfonamidoC₁₋₄alkyl, heterocyclyl, aryl, arylC₁₋₄alkoxy, aryloxy, arylthio, arylmethyl, aroyl, aryloxymethyl, arylsulfonyl, aryl-NR′— (wherein R′ is hydrogen or C₁₋₄alkyl), arylsulfonyloxy, arylsulfonylC₁₋₄alkyl, arylsulfonamido, arylcarboxamido, arylsulfonamidoC₁₋₄alkyl, arylcarboxamidoC₁₋₄alkyl, aroylC₁₋₄alkyl, arylC₁₋₄alkanoyl, a group R₁₁CON(R₁₂)(CH₂)ᵣ, R₁₁R₁₂NCO(CH₂)ᵣ or R₁₁R₁₂NSO₂(CH₂)ᵣ (in which r is 0, 1, 2, 3 or 4, and each of R₁₁ and R₁₂ is independently hydrogen or C₁₋₄alkyl, or in the groups R₁₁CON(R₁₂)(CH₂)ᵣ, R₁₁R₁₂NCO(CH₂)ᵣ and R₁₁R₁₂NSO₂(CH₂)ᵣ, R₁₁CONR₁₂ or R₁₁R₁₂N together form a 4-, 5-, 6- or 7-membered azacyclic group optionally containing one additional O, N or S atom in the azacycle and having 3-8 carbon atoms (including the carbon atoms contained in any optional substituent(s) of the azacycle)); wherein in any group containing an aryl moiety, the aryl may be substituted by one, two or three groups selected from the group consisting of halogen, hydroxy, cyano, nitro, amino, C₁₋₄alkyl, haloC₁₋₄alkyl, C₁₋₄alkoxy, haloC₁₋₄alkoxy, C₁₋₄alkylenedioxy, C₁₋₄alkanoyl, C₁₋₄alkylsulfonyl, haloC₁₋₄alkylsulfonyl, C₁₋₄alkylamino, C₁₋₄dialkylamino, R₁₃R₁₄NCO (in which R₁₃ and R₁₄ are independently hydrogen or C₁₋₄alkyl, or R₁₃R₁₄N together form a 4-, 5-, 6- or 7-membered azacyclic group optionally containing one additional O, N or S atom in the azacycle and having 3-8 carbon atoms (including the carbon atoms contained in any optional substituent(s) of the azacycle));
A and B are independently N or CH;
R₅, R₆, R₇, R₈ and R₉ are independently hydrogen or C₁₋₄alkyl;
R₁₀ is a group of the formula (a) or (b):

—Z (a)

—(CR₁₅R₁₆)ₜZ (b)
wherein:
   Z is C₁₋₄alkyl, haloC₁₋₄alkyl, C₃₋₆cycloalkyl, phenyl, heterocyclyl, a 5- or 6-membered heteroaromatic ring or a 8- to 11-membered bicyclic group, any of which is optionally substituted by 1, 2, 3 or 4 substituents selected from the group consisting of: halogen, hydroxy, oxo, cyano, nitro, C₁₋₄alkyl, C₁₋₄alkoxy, haloC₁₋₄alkyl, haloC₁₋₄alkoxy, C₁₋₄alkylenedioxy, C₁₋₄alkanoyl, C₁₋₄alkylsulfonyl, C₁₋₄alkylsulfonyloxy, haloC₁₋₄alkylsulfonyl, haloC₁₋₄alkylsulfonyloxy, C₁₋₄alkylsulfinyl, C₁₋₄alkylthio, R₁₇SO₂N(R₁₈)—, R₁₇R₁₈NSO₂—, R₁₇R₁₈N—, R₁₇R₁₈NCO—, R₁₇CONR₁₈— and a 5- or 6-membered heteroaromatic ring which is optionally substituted by one or two C₁₋₂alkyl, haloC₁₋₂alkyl or R₁₇R₁₈N—(wherein R₁₇ and R₁₈ are independently hydrogen or C₁₋₄alkyl, or R₁₇ and R₁₈ together form C₃₋₆alkylene); and wherein substituents positioned ortho to one another may be linked to form a 5- or 6-membered ring; and
   R₁₅ and R₁₆ are independently hydrogen or C₁₋₄alkyl and t is 1, 2, 3 or 4, or —(CR₁₅R₁₆)ₜ— forms a C₃₋₆cycloalkylene linker.

In formula (I), “—S—” means thio (sulfur).

The term “C₁₋₄alkyl” refers to an alkyl group having from one to four carbon atoms, in all isomeric forms, such as methyl, ethyl, propyl, isopropyl, butyl, isobutyl, sec-butyl and tert-butyl.

The term “alkylene” refers to a straight or branched chain divalent hydrocarbon radical. Examples of C₁₋₃alkylene groups include methylene, ethylene and n-propylene. Examples of “C₁₋₄alkylene” include, in addition to the above, n-butylene.

The term “C₁₋₄alkoxy” refers to a straight chain or branched chain alkoxy (or “alkyloxy”) group having from one to four carbon atoms, such as methoxy, ethoxy, propoxy, isopropoxy, butoxy, isobutoxy, sec-butoxy and tert-butoxy.

The term “halogen” and its abbreviation “halo” refer to fluorine (F), chlorine (Cl), bromine (Br) or iodine (1). Where the term “halo” is used before another group, it indicates that the group is substituted by one, two or three halogen atoms. For example, “haloC₁₋₄alkyl” refers to groups such as trifluoromethyl, bromoethyl, trifluoropropyl, and other groups derived from C₁₋₄alkyl groups as defined above; and the term “haloC₁₋₄alkoxy” refers to groups such as trifluoromethoxy, bromoethoxy, trifluoropropoxy, and other groups derived from C₁₋₄alkoxy groups as defined above.

The term “C₁₋₄alkoxyC₁₋₄alkyl” refers to a C₁₋₄alkoxy group attached through a C₁₋₄alkylene group, for example methoxymethyl, ethoxymethyl, propoxyethyl, isopropoxyethyl and others derived from the C₁₋₄alkoxy and C₁₋₄alkyl groups as defined above.

The term “C₁₋₄alkylthio” refers to a C₁₋₄alkyl group attached through a sulfur atom (—S—). Examples of C₁₋₄alkylthio include methylthio, ethylthio, propylthio and butylthio.

The term “C₃₋₆cycloalkyl” refers to a cycloalkyl group having from three to six carbon atoms, such as cyclopropyl, cyclobutyl, cyclopentyl and cyclohexyl. The term “C₃₋₆cycloalkylene” refers to a divalent cycloalkyl group, such as cyclopropylene, cyclobutylene, cyclopentylene and cyclohexylene.

The term “C₃₋₆cycloalkylC₁₋₄alkyl” refers to a cycloalkyl group attached through a C₁₋₄alkylene group, such as cyclopropylmethyl, cyclobutylethyl, and others derived from C₃₋₆cycloalkyl groups and C₁₋₄alkyl groups as defined above.

The term “aryl” refers to phenyl or a 5- or 6-membered heteroaromatic ring. Examples of 5- or 6-membered heteroaromatic rings include furanyl, thienyl, pyrrolyl, oxazolyl, thiazolyl, imidazolyl, oxadiazolyl, thiadiazolyl, pyridinyl, triazolyl, triazinyl, pyridazyl, pyrimidinyl, isothiazolyl, isoxazolyl, pyrazinyl, pyrazolyl and pyrimidinyl.

The term “arylC₁₋₄alkyl” refers to an aryl group attached through a C₁₋₄alkylene group. The C₁₋₆alkylene group may be in any suitable isomeric form. Examples of arylC₁₋₄alkyl include benzyl, phenethyl (including phenyl-CH₂CH₂— and phenyl-C(CH₃)—) and others derived from the aryl groups and C₁₋₄alkyl groups as defined above.

The terms “arylC₁₋₄alkoxy” refers to an aryl group attached through a C₁₋₄alkoxy group. Examples of arylC₁₋₄alkoxy include benzyloxy (phenyl-CH₂O—) and phenylethoxy.

The term “sulfonyl” refers to the group —SO₂—. Thus, the term “C₁₋₄alkylsulfonyl” includes methylsulfonyl, ethylsulfonyl, and others derived from the C₁₋₄alkyl groups defined above.

The term “haloC₁₋₄alkylsulfonyl” refers to groups such as trifluoromethanesulfonyl and pentafluoroethylsulfonyl. The term “arylsulfonyl” includes phenylsulfonyl, pyridinylsulfonyl, and others derived from aryls as defined above.

The term “arylcarboxamido” refers to groups such as phenylcarboxamido and pyridinylcarboxamido, and others derived from the aryl groups as defined above.

The term “C₁₋₄alkylenedioxy” refers to groups such as methylenedioxy, ethylenedioxy and others derived from C₁₋₄alkyl as defined above.

The term “5- or 6-membered heteroaromatic ring” refers to a monocyclic 5- or 6-membered heterocyclic group containing 1, 2, 3 or 4 heteroatoms, for example from 1 to 3 heteroatoms, selected from O, N and S. When the group contains 2-4 heteroatoms, one may be selected from O, N and S and the remaining heteroatoms may be N. Examples of 5 and 6-membered heteroaromatic rings include pyrrolyl, pyrrolinyl, pyrazolinyl, imidazolyl, pyrazolyl, oxazolyl, isoxazolyl, oxadiazolyl, isothiazolyl, thiazolyl, furyl, thienyl, thiadiazolyl, pyridyl, triazolyl, thiazinyl, triazinyl, pyridazinyl, pyrimidinyl and pyrazinyl.

The term “8- to 11-membered bicyclic group” refers to a bicyclic ring system containing a total of 8, 9, 10 or 11 carbon atoms, wherein 1, 2, 3 or 4 or 5 of the carbon atoms are optionally replaced by a heteroatom independently selected from O, S and N. The term includes bicyclic systems wherein both rings are aromatic, as well as bicyclic ring systems wherein one of the rings is partially or fully saturated. Examples of 8- to 11-membered bicyclic groups wherein both rings are aromatic include indenyl, naphthyl and azulenyl. Examples of 8- to 11-membered bicyclic groups having 1, 2, 3, 4 or 5 heteroatoms, in which both rings are aromatic, include: 6H-thieno[2,3-b]pyrrolyl, imidazo[2,1-b][1,3]thiazolyl, imidazo[5,1-b][1,3]thiazolyl, [1,3]thiazolo[3,2-b][1,2,4]triazolyl, indolyl, isoindolyl, indazolyl, benzimidazolyl e.g. benzimidazol-2-yl, benzoxazolyl e.g. benzoxazol-2-yl, benzisoxazolyl, benzothiazolyl, benzisothiazolyl, benzothienyl, benzofuranyl, naphthridinyl, quinolyl, quinoxalinyl, quinazolinyl, cinnolinyl and isoquinolyl. Examples of 8- to 11-membered bicyclic groups having 1, 2, 3, 4 or 5 heteroatoms, in which one of the rings is partially or fully saturated includes dihydrobenzofuranyl, indanyl, tetrahydronaphthyl, indolinyl, isoindolinyl, tetrahydroisoquinolinyl, tetrahydroquinolyl, benzoxazinyl and benzoazepinyl.

The term “heterocyclyl” refers to a 5 or 6-membered monocyclic or 8 to 1′-membered bicyclic group wherein 1, 2, 3, 4 or 5 of the carbon atoms are replaced by a heteroatom independently selected from O, S and N and which is partially or fully saturated. Examples of “heterocyclyl” which are fully saturated 5 or 6-membered monocyclic rings include pyrrolidinyl, imidazolidinyl, pyrazolidinyl, isothiazolyl, thiazolyl, tetrahydrofuranyl, dioxolanyl, piperidinyl, piperazinyl, morpholinyl, thiomorpholinyl, tetrahydrothienyl, dioxanyl, tetrahydro-2H-pyranyl and dithianyl. Examples of “heterocyclyl” groups which are partially saturated 5 or 6-membered monocyclic rings include oxazolinyl, isoaxazolinyl, imidazolinyl, pyrazolinyl, 1,2,3,6-tetrahydropyridyl and 3,6-dihydro-2H-pyranyl. Examples of “heterocyclyl” groups which are fully saturated 8 to 11-membered bicyclic rings include decahydroquinolinyl, octahydro-2H-1,4-benzoxazinyl and octahydro-1H-cyclopenta[b]pyridinyl. Examples of “heterocyclyl” groups which are partially saturated 8 to 11-membered bicyclic rings include 2,3-dihydro-1H-indolyl, 1,2,3,4-tetrahydroquinolinyl, 1,2,3,4-tetrahydroisoquinolinyl and 2,3,4,5-tetrahydro-1H-3-benzazepinyl.

Any of these groups may be attached to the rest of the molecule at any suitable position.

As used herein, the term “salt” refers to any salt of a compound according to the present invention prepared from an inorganic or organic acid or base, quaternary ammonium salts and internally formed salts. Physiologically acceptable salts are suitable for medical applications because of their greater aqueous solubility relative to the parent compounds. Such salts must clearly have a physiologically acceptable anion or cation. Suitably physiologically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example benzenesulfonic and p-toluenesulfonic, acids; base addition salts formed with alkali metals and alkaline earth metals and organic bases such as N,N-dibenzylethylenediamine, chloroprocaine, choline, diethanolamine, ethylenediamine, meglumaine (N-methylglucamine), lysine and procaine; and internally formed salts. Salts having a non-physiologically acceptable anion or cation are within the scope of the invention as useful intermediates for the preparation of physiologically acceptable salts and/or for use in non-therapeutic, for example, in vitro, situations.

When R₂ or R₃ contains an aryl moiety, ie R₂ or R₃ is aryl, arylc₁₄alkoxy, aryloxy, arylthio, arylmethyl, aroyl, aryloxymethyl, arylsulfonyl, aryl-NR′—, arylsulfonyloxy, arylsulfonylc₁₋₄alkyl, arylsulfonamido, arylcarboxamido, arylsulfonamidoC₁₋₄alkyl, arylcarboxamidoC₁₋₄alkyl, aroylC₁₋₄alkyl or arylC₁₋₄alkanoyl, the aryl moiety is optionally substituted by one or two substituents selected from: halogen, cyano, C₁₋₂alkyl (e.g. methyl), fluoroC₁₋₂alkyl (eg trifluoromethyl), C₁₋₂alkoxy (e.g. methoxy), C₁₋₂alkylenedioxy (e.g. methylenedioxy), C₁₋₃alkanoyl (e.g. acetyl), C₂alkanoylamino (e.g. acetylamino), fluoroC₁alkylsulfonyl (e.g. trifluoromethylsulfonyl) and methylsulfonyl. For example, the aryl moiety is optionally substituted by one or two methyl.

When R₂ or R₃ is a group R₁₁CON(R₁₂)(CH₂)ᵣ, R₁₁R₁₂NCO(CH₂)ᵣ or R₁₁R₁₂NSO₂(CH₂)ᵣ and R₁₁CONR₁₂ or R₁₁R₁₂N together form a 4-, 5-, 6- or 7-membered azacyclic group, then this is characterised by: (i) containing one additional O, N or S atom in the azacycle, for example the azacyclic group being 1,4-morpholin-4-yl and/or (ii) having 1 or 2 optional C₁₋₂alkyl substituents whose carbon atoms are included in the azacyclic group's 3-8 carbon atoms. One, two or more F atoms can optionally be included as substituents of the carbon atoms of the heterocycle. The term “azacyclic group” should be interpreted to cover only stable azacycles such as 1,4-morpholine and piperazine and not for example 1,3-morpholine. In one aspect the present invention provides saturated azacycles, e.g. piperidinyl, pyrrolidinyl, 1,4-morpholinyl, and including the corresponding α-oxo-azacycles R₁₁CONR₁₂.

In one embodiment, R₂ or R₃ is halogen, cyano, acetyl, trifluoromethyl, pentafluoroethyl, trifluoromethoxy, C₁₋₄alkylsulfonyl, C₁₋₄alkylsulfonyloxy, R₁₁R₁₂NSO₂ (where each of R₁₁ and R₁₂ is independently hydrogen or C₁₋₄alkyl or R₁₁R₁₂N together form a 4-, 5-, 6- or 7-membered azacyclic group optionally containing one additional O, N or S atom in the azacycle and having 3-8 carbon atoms), a heterocyclyl, or a 5- or 6-membered heteroaromatic ring which is optionally substituted by one or two substituents selected from: halogen, cyano, C₁₋₂alkyl (e.g. methyl), haloC₁₋₂alkyl (e.g. trifluoromethyl), C₁₋₂alkoxy (e.g. methoxy), C₁₋₂alkylenedioxy (e.g. methylenedioxy), C₁₋₃alkanoyl (e.g. acetyl), C₂alkanoylamino (e.g. acetylamino), haloc₁alkylsulfonyl (e.g. trifluoromethylsulfonyl) and methylsulfonyl.

In one embodiment, R₃ is hydrogen.

Examples of R₂ include: C₁₋₄alkyl, haloC₁₋₄alkyl, halogen, C₁₋₄alkylsulfonyl (e.g. methylsulfonyl or ethylsulfonyl), haloC₁₋₄alkylsulfonyl (e.g. trifluoromethylsulfonyl), C₁₋₄alkylsulfonyloxy (e.g. methylsulfonyloxy), haloC₁₋₄alkylsulfonyloxy (e.g. trifluoromethylsulfonyloxy), R₁₁R₁₂NSO₂ (where each of R₁₁ and R₁₂ is independently hydrogen or C₁₋₄alkyl or R₁₁R₁₂N together form a 4-, 5-, 6- or 7-membered azacyclic group optionally containing one additional O, N or S atom in the azacycle and having 3-8 carbon atoms, e.g. a piperidin-1-ylsulfonyl, pyrrolidin-1-ylsulfonyl or 1,4-morpholin-4-ylsulfonyl), a 5- or 6-membered heteroaromatic or a heterocyclyl, each of which is optionally substituted by one or two substituents selected from: halogen, cyano, C₁₋₂alkyl (e.g. methyl or trifluoromethyl), C₁₋₂alkoxy (e.g. methoxy), C₁₋₂alkylenedioxy (e.g. methylenedioxy), C₁₋₃alkanoyl (e.g. acetyl), C₂alkanoylamino (e.g. acetylamino), haloC₁alkylsulfonyl (e.g. trifluoromethylsulfonyl) and methylsulfonyl.

Suitably, R₂ is bromo, cyano, hydroxy, chloro, methoxy, tert-butyl, methylsulfonyl, ethylsulfonyl, N,N-dimethylaminosulfonyl, pyrrolidin-1-ylsulfonyl, 1,4-morpholin-4-ylsulfonyl, methylsulfonyloxy, pyrazolyl (eg pyrazol-5-yl), 1,3-dimethyl-pyrazol-5-yl, pyrazin-2-yl, 5-methyl-oxazol-2-yl or 5-methyl-isoxazol-3-yl.

In one embodiment, at least one of R₁ and R₄ is hydrogen. For example, both R₁ and R₄ are hydrogen, or all of R₁, R₃ and R₄ are hydrogen.

In one embodiment, at least one of A and B is nitrogen. For example, A and B may both be nitrogen.

In one embodiment, R₅, R₆, R₇ and R₈ are all hydrogen.

In one embodiment, R₉ is methyl.

R₁₀ may be formula (a) or (b). For formula (a) and (b), in one embodiment, Z may be optionally substituted phenyl such as 3,4-difluorophenyl, an optionally substituted monocyclic group such as pyrazinyl (eg 2-pyrazinyl), or an optionally substituted bicyclic group such as quinolinyl (e.g. 2-, 3-, 4-, 5- or 6-quinolinyl), 4-tetrahydro-2H-pyranyl, furyl (e.g. 2-furyl), thienyl (e.g. 2-thienyl), pyridyl (e.g. 4-pyridyl), indolyl, pyrazolopyrimidyl (e.g. pyrazolo[1,5-a]pyrimidyl), cinnolinyl, benzo[b]furanyl, thienopyridine or pyrrolopyridyl. Examples of Z include 4-tetrahydro-2H-pyranyl, 4-trifluoromethylphenyl, furyl (e.g. 2-furyl), thienyl (e.g. 2-thienyl), pyridyl (e.g. 4-pyridyl), 2-methylquinolinyl (e.g. 2-methylquinolin-5-yl), 5-methyl-2-pyrazinyl, 3,4-difluorophenyl, and 4-methyl,3-oxazol-5-yl.

When R₁₀ is a group of formula (b), and R₁₂ and R₁₃ are independently hydrogen or C₁₋₄alkyl and t is 1, 2, 3 or 4, examples include —(CH₂)-Z, and —(CHCH₃)-Z. When the group —(CR₁₅R₁₆)ₜ— in formula (b) forms a C₃₋₆cycloalkylene linker, examples include groups such as:

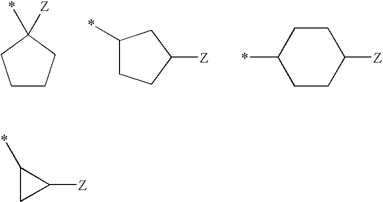

In one embodiment, Z is unsubstituted or substituted by one or more substituents selected from: halogen, or cyano, C₁₋₂alkyl (e.g. methyl), haloC₁₋₂alkyl (e.g. trifluoromethyl), C₁₋₂alkoxy (e.g. methoxy), haloC₁₋₄alkoxy (e.g. trifluoromethoxy), C₁₋₂alkylenedioxy (e.g. methylenedioxy), C₂₋₃alkanoyl (e.g. acetyl), C₂alkanoylamino (e.g. acetylamino), methylsulfonyl, haloC₁ alkylsulfonyl (e.g. trifluoromethylsulfonyl), C₁ alkylsulfonyloxy (e.g. methylsulfonyloxy), C₁alkylaminosulfonyl (e.g. methylaminosulfonyl), C₁alkylsulfonylamino (e.g. methylsulfonylamino) and C₁alkylaminocarbonyl (e.g. methylaminocarbonyl).

In one embodiment, R₁₀ is a group of formula (a) as defined in formula (I). For example, R₁₀ may be optionally substituted phenyl, such as unsubstituted phenyl or fluorophenyl (e.g. 4-fluorophenyl), or optionally substituted quinolinyl (e.g. 6-quinolinyl).

In one embodiment, a compound of formula (IA) or a salt thereof is provided:

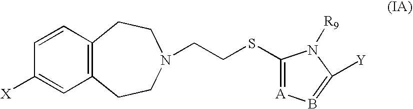

wherein
A, B and R₉ are as defined for formula (I);
X is a 5- or 6-membered heteroaromatic ring optionally substituted by 1, 2 or 3 substituents selected from the group consisting of: halogen, cyano, C₁₋₂alkyl, fluoroC₁₋₂alkyl, C₁₋₂alkoxy, C₁₋₃alkanoyl, C₂alkanoylamino, fluoroc₁alkylsulfonyl and methylsulfonyl; and
Y is phenyl, heterocyclyl, a 5- or 6-membered heteroaromatic ring or a 8- to 11-membered bicyclic group, any of which is optionally substituted by 1, 2, 3 or 4 substituents selected from the group consisting of: halogen, cyano, C₁₋₂alkyl, haloC₁₋₂alkyl, C₁₋₂alkoxy, haloC₁₋₂alkoxy, C₁₋₂alkylenedioxy, C₂₋₃alkanoyl, C₂alkanoylamino, methylsulfonyl, haloC₁alkylsulfonyl, methylsulfonyloxy, methylaminosulfonyl, methylsulfonylamino and methylaminocarbonyl.

All embodiments and features of formula (I) apply to formula (IA).

In another embodiment, a compound of formula (IB) or a salt thereof is provided:

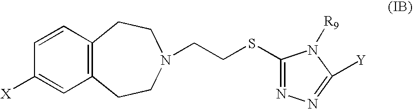

wherein
X is isoxazolyl or pyrazolyl ring optionally substituted by 1, 2 or 3 substituents selected from the group consisting of: halogen, cyano, C₁₋₂alkyl, fluoroC₁₋₂alkyl, C₁₋₂alkoxy, C₁₋₃alkanoyl, C₂alkanoylamino, fluoroc₁alkylsulfonyl and methylsulfonyl; and
Y is phenyl, heterocyclyl, a 5- or 6-membered heteroaromatic ring or a 8- to 11-membered bicyclic group, any of which is optionally substituted by 1, 2, 3 or 4 substituents selected from the group consisting of: halogen, cyano, C₁₋₂alkyl, haloC₁₋₂alkyl, C₁₋₂alkoxy, haloC₁₋₂alkoxy, C₁₋₂alkylenedioxy, C₂₋₃alkanoyl, C₂alkanoylamino, methylsulfonyl, haloC₁alkylsulfonyl, methylsulfonyloxy, methylaminosulfonyl, methylsulfonylamino and methylaminocarbonyl.

All embodiments and features of formula (I) apply to formula (IB).

Example compounds of the present invention include:
1. 7-(5-Methyl-3-isoxazolyl)-3-(2-{[4-methyl-1,3-oxazol-5yl)-4H-1,2,4-triazol-3-yl]thio}ethyl)-2,3,4,5-tetrahydro-1H-3-benzazepine
2. 7-(5-Methyl-3-isoxazolyl)-3-(2-{[4-methyl-5-(tetrahydro-2H-pyran-4-yl)-4H-1,2,4-triazol-3-yl]thio}ethyl)-2,3,4,5-tetrahydro-1H-3-benzazepine
3. 7-(5-Methyl-3-isoxazolyl)-3-(2-{[4-methyl-5-(2-methyl-5-quinolinyl)-4H-1,2,4-triazol-3-yl]thio}ethyl)-2,3,4,5-tetrahydro-1H-3-benzazepine
4. 7-(5-Methyl-3-isoxazolyl)-3-(2-{[4-methyl-5-(2-methyl-6-quinolinyl)-4H-1,2,4-triazol-3-yl]thio}ethyl)-2,3,4,5-tetrahydro-1H-3-benzazepine
5. 7-(1,3-Dimethyl-1H-pyrazol-5-yl)-3-(2-{[4-methyl-5-(2-methyl-5-quinolinyl)-4H-1,2,4-triazol-3-yl]thio}ethyl)-2,3,4,5-tetrahydro-1H-3-benzazepine
6. 7-(1,3-Dimethyl-1H-pyrazol-5-yl)-3-(2-{[4-methyl-5-(5-methyl-2-pyrazinyl)-4H-1,2,4-triazol-3-yl]thio}ethyl)-2,3,4,5-tetrahydro-1H-3-benzazepine
7. 3-(2-{[5-(3,4-Difluorophenyl)-4-methyl-4H-1,2,4-triazol-3-yl]thio}ethyl)-7-(1,3-dimethyl-1H-pyrazol-5-yl)-2,3,4,5-tetrahydro-1H-3-benzazepine
8. 7-(5-Methyl-3-isoxazolyl)-3-(2-{[4-methyl-5-(2-methyl-3-pyridinyl)-4H-1,2,4-triazol-3-yl]thio}ethyl)-2,3,4,5-tetrahydro-1H-3-benzazepine formate
9. 7-(5-Methyl-3-isoxazolyl)-3-(2-{[4-methyl-5-(4-pyridazinyl)-4H-1,2,4-triazol-3-yl]thio}ethyl)-2,3,4,5-tetrahydro-1H-3-benzazepine formate
10. 7-(5-Methyl-3-isoxazolyl)-3-[2-({4-methyl-5-[2-methyl-6-(trifluoromethyl)-3-pyridinyl]-4H-1,2,4-triazol-3-yl}thio)ethyl]-2,3,4,5-tetrahydro-1H-3-benzazepine formate
11. 3-(2-{[5-(1,5-Dimethyl-1H-pyrazol-4-yl)-4-methyl-4H-1,2,4-triazol-3-yl]thio}ethyl)-7-(5-methyl-3-isoxazolyl)-2,3,4,5-tetrahydro-1H-3-benzazepine formate
12. 3-(2-{[5-(5-Chloro-1-methyl-1H-pyrazol-4-yl)-4-methyl-4H-1,2,4-triazol-3-yl]thio}ethyl)-7-(5-methyl-3-isoxazolyl)-2,3,4,5-tetrahydro-1H-3-benzazepine formate
13. 7-(5-Methyl-3-isoxazolyl)-3-[2-({4-methyl-5-[4-(trifluoromethyl)phenyl]-4H-1,2,4-triazol-3-yl}thio)ethyl]-2,3,4,5-tetrahydro-1H-3-benzazepine formate
14. 3-(2-{[5-(3,4-Difluorophenyl)-4-methyl-4H-1,2,4-triazol-3-yl]thio}ethyl)-7-(5-methyl-3-isoxazolyl)-2,3,4,5-tetrahydro-1H-3-benzazepine formate
15. 7-(5-Methyl-3-isoxazolyl)-3-(2-{[4-methyl-5-(5-methyl-2-pyrazinyl)-4H-1,2,4-triazol-3-yl]thio}ethyl)-2,3,4,5-tetrahydro-1H-3-benzazepine formate
16. 3-(2-{[1-(1-Methylethyl)-5-(methylsulfonyl)-1H-benzimidazol-2-yl]thio}ethyl)-7-(5-methyl-3-isoxazolyl)-2,3,4,5-tetrahydro-1H-3-benzazepine formate and pharmaceutically acceptable salts thereof.

It will be appreciated that for use in medicine the salts of the compounds of the invention should be pharmaceutically (i.e physiologically) acceptable. Suitable pharmaceutically acceptable salts will be apparent to those skilled in the art and include for example acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Other non-pharmaceutically acceptable salts eg. oxalates, may be used, for example in the isolation of compounds of the invention and are included within the scope of this invention. Also included within the scope of the invention are solvates, hydrates, complexes and prodrugs of compounds of the invention.

Certain of the compounds of the invention may form acid addition salts with less than one equivalent of the acid, or one or more equivalents of the acid. The present invention includes within its scope all possible stoichiometric and non-stoichiometric forms.

Certain groups/substituents included in the present invention may be present as isomers. The present invention includes within its scope all such isomers, including racemates, enantiomers, tautomers and mixtures thereof. Certain of the substituted heteroaromatic rings included in compounds of formula (I) may exist in one or more tautomeric forms. The present invention includes within its scope all such tautomeric forms, including mixtures.

In one aspect the present invention provides compounds having a molecular weight of 800 or less. In another aspect the present invention provides compounds having a molecular weight of 600 or less. Generally, and without being limited thereto, such compounds may have higher oral bioavailability, and sometimes higher solubility and/or brain penetrancy. Molecular weight here refers to that of the unsolvated free base compound, excluding any molecular weight contributed by addition salts, solvent (e.g. water) molecules, prodrug molecular parts cleaved off in vivo, etc.

In general, the compounds or salts of the invention should be interpreted as excluding those compounds (if any) which are so chemically unstable, either per se or in water, that they are clearly unsuitable for pharmaceutical use through all administration routes, whether oral, parenteral or otherwise. Such compounds are known to the skilled chemist. Prodrugs or compounds which are stable ex vivo and which are convertable in the mammalian (e.g. human) body to the inventive compounds are however included.

The present invention also provides a process for preparing a compound of formula (I), which process comprises:

(a) reacting a compound of formula (II):

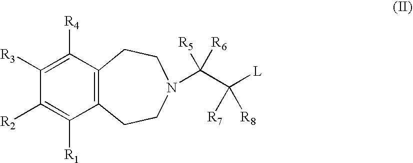

wherein R₁ to R₈ are as defined for formula (I) and L is a leaving group; with a compound of formula (III):

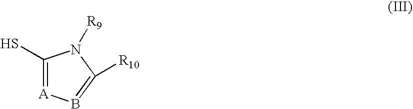

wherein A, B, R₉ and R₁₀ are as defined for formula (I); or

(b) for a compound of formula (I) wherein R₂ is aryl, reacting a compound of formula (IV):

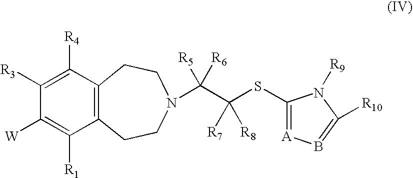

wherein R₁, R₃ to R₁₀, A and B are as defined for formula (I) and W is halogen or a trifluoromethylsulfonyloxy group, or W is a group M selected from a boron derivative (e.g. a boronic acid function B(OH)₂) or a metal function such as trialkylstannyl (e.g. SnBu₃), zinc halide or magnesium halide; with a compound aryl-W¹, wherein aryl is as defined for formula (I), W¹ is halogen or a trifluoromethylsulfonyloxy group when W is a group M or W¹ is a group M as defined above when W is halogen or a trifluoromethylsulfonyloxy group; or

(c) for a compound of formula (I) wherein R₂ is aryloxy or arylthio, reacting a compound of formula (V):

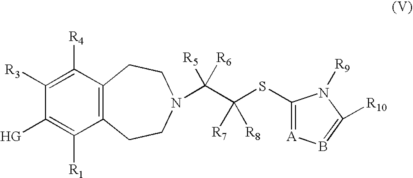

wherein G is oxygen or sulfur, and R₁, R₃ to R₁₀, A and B are as defined for formula (I); with a reagent serving to introduce the aryl group;

and optionally thereafter for any of the steps (a), (b) or (c):
removing any protecting group(s); and/or
forming a salt; and/or
converting one compound of formula (I) to a different compound of formula (I).

Process (a) may be effected using conventional methods for the formation of a thioether. The leaving group L can be halogen such as chlorine. Alternatively L can be a sulfonyloxy group such C₁₋₄alkylsulfonyloxy (e.g. methanesulfonyloxy or trifluoromethanesulfonyloxy); or AR′-sulfonyloxy wherein AR′ is optionally substituted phenyl, an optionally substituted 5- or 6-membered aromatic heterocyclic group, or an optionally substituted bicyclic group, preferably optionally substituted phenyl, wherein in each case the optional substituents are one or more C₁₋₂alkyl groups; e.g. para-toluenesulfonyloxy. When L is a halogen the reaction may be carried out using a base such as lithium hydroxide in a solvent such as N,N-dimethylformamide.

The reaction in process (b), and the reaction in process (d), may be effected in the presence of a transition metal e.g., palladium catalyst such as bis-triphenylphosphinepalladium dichloride or tetrakis-triphenylphosphinepalladium (0). When M is a boronic acid function such as B(OH)₂ the reaction may be carried out under basic conditions, for example using aqueous sodium carbonate in a suitable solvent such as dioxane. When M is trialkylstannyl the reaction may be carried out in an inert solvent, such as xylene or dioxane optionally in the presence of LiCl. When M is a zinc or magnesium halide the reaction may be effected in an aprotic solvent such as tetrahydrofuran. The substituent W is preferably halogen such as bromine, or a sulfonyloxy group such as trifluoromethylsulfonyloxy; and W¹ is preferably a group M, such as trialkylstannyl or B(OH)₂.

In process (c), the reagent serving to introduce the aryl group is preferably a compound of formula aryl-Hal, wherein Hal is halogen. The reaction may be effected in the presence of a base, such as potassium carbonate, in a solvent such as N,N-dimethylformamide.

A compound of formula (II) may itself be prepared by reacting a compound of formula (VII):

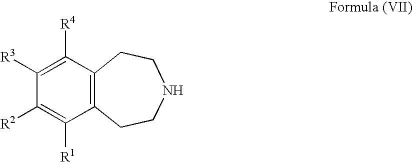

wherein R¹ to R⁴ are as hereinbefore defined; with a compound of formula (VIII):

L′CH₂CH₂L Formula (VIII)

wherein L is as herein defined and L′ is a leaving group, e.g., a bromine atom or alternatively with a compound of formula (IX)

OHCCH₂L Formula (IX)

wherein L is as herein defined, in the presence of a hydride source such as sodium triacetoxyborohydride.

Compounds of formula (I) may be converted to another compound of formula (I) by suitable methods known to the skilled person, such as:

(i) converting one or more of R₁ to R₄ from alkoxy (e.g. methoxy) to hydroxyl; and
(ii) converting one or more of R₂ or R₃ from hydroxy to sulfonyloxy, such as alkylsulfonyloxy e.g. methanesulfonyloxy or trifluoromethanesulfonyloxy.

Compounds of formula (I) have been found to exhibit affinity for dopamine receptors, in particular the D₃ receptor, and are expected to be useful in the treatment of disease states which require modulation of such receptors, such as psychotic conditions.

Such affinity is typically calculated from the IC₅₀ as the concentration of a compound necessary to displace 50% of the radiolabeled ligand from the receptor, and is reported as a “Kᵢ” value calculated by the following equation:

$${K}_{i} = \frac{{IC}_{50}}{1 + L / {K}_{D}}$$

where L=radioligand and K_{D}=affinity of radioligand for receptor (Cheng and Prusoff, Biochem. Pharmacol. 22:3099, 1973).

In the context of the present invention pKi (corresponding to the antilogarithm of Ki) is used instead of Ki and the compounds of the present invention typically show pKi greater than 7. In one aspect the present invention provides compounds of formula (I) having a pKi comprised between 7 and 8. In another aspect the present invention provides compounds of formula (I) having a pKi comprised between 8 and 9. In a further aspect the present invention provides compounds of formula (I) having a pKi greater than 9.

Many of the compounds of formula (I) have also been found to have greater affinity for dopamine D₃ than for D₂ receptors. Compounds for formula (I) have also been found to exhibit low affinity for the H1 receptor. A low affinity for the H1 receptor generally leads to avoidance of: (1) sedation, somnolence, and fatigue; (2) cardiotoxicity; (3) potentiation of opioid-induced sedation and respiratory depression; (4) short-term weight gain; (5) impaired cognition (memory, spatial cognition, attention, tracking performance); (6) impaired psychomotor performance including quick tolerance to these effects, and (7) altered neuroendocrine regulation of prolactin and potentially other hormones.

The therapeutic effect of currently available antipsychotic agents (neuroleptics) is generally believed to be exerted via blockade of D₂ receptors; however this mechanism is also thought to be responsible for undesirable extrapyramidal side effects (eps) associated with many neuroleptic agents. Without wishing to be bound by theory, it has been suggested that blockade of the more recently characterised dopamine D₃ receptor may give rise to beneficial antipsychotic activity without significant eps. (see for example Sokoloff et al, Nature, 1990; 347: 146-151; and Schwartz et al, Clinical Neuropharmacology, Vol 16, No. 4, 295-314, 1993). In one embodiment compounds of the present invention are provided which have higher (e.g. ≧10× or ≧100× higher) affinity for dopamine D₃ than dopamine D₂ receptors (such affinity can be measured using standard methodology for example using cloned dopamine receptors—see herein). Said compounds may suitably be used as selective modulators of D₃ receptors.

Compounds of formula (I) will be used for treatment of all aspects of drug dependency including prevention of relapse to and relief of withdrawal symptoms from drugs of abuse such as nicotine, alcohol, cocaine, amphetamine, metamphetamine, opiates, benzodiazepines, inhalants and inhibition of tolerance induced by opioids. In addition, compounds of formula (I) and pharmaceutically acceptable salts and solvates thereof will be used to reduce craving and therefore will be useful in the treatment of drug craving. Drug craving can be defined as the incentive motivation to self-administer a psychoactive substance that was previously consumed. Three main factors are involved in the development and maintenance of drug craving: (1) Dysphoric states during drug withdrawal can function as a negative reinforcer leading to craving; (2) Environmental stimuli associated with drug effects can become progressively more powerful (sensitization) in controlling drug seeking or craving, and (3) A cognition (memory) of the ability of drugs to promote pleasurable effects and to alleviate a dysphoric state during withdrawal. Craving may account for the difficulty that individuals have in giving up drugs of abuse and therefore contributes significantly to the maintenance of drug dependence and the probability of relapse or reinstatement of drug seeking and drug taking behaviors.

The compounds of formula (I) are of potential use as antipsychotic agents for example in the treatment of schizophrenia, schizo-affective disorders, psychotic depression, mania, paranoid and delusional disorders. Furthermore, they could have utility as adjunct therapy in Parkinsons Disease, particularly with compounds such as L-DOPA and possibly dopaminergic agonists, to reduce the side effects experienced with these treatments on long term use (e.g. see Schwartz et al., Brain Res. Reviews, 1998, 26, 236-242). From the localisation of D3 receptors, it could also be envisaged that the compounds could also have utility for the treatment of substance abuse where it has been suggested that D3 receptors are involved (e.g. see Levant, 1997, Pharmacol. Rev., 49, 231-252). Examples of such substance abuse include alcohol, cocaine, heroin and nicotine abuse. Other conditions which may be treated by the compounds include dyskinetic disorders such as Parkinson's disease, neuroleptic-induced parkinsonism and tardive dyskinesias; depression; anxiety, cognitive impairment including memory disorders such as Alzheimers disease, eating disorders, sexual dysfunction, sleep disorders, emesis, movement disorders, obsessive-compulsive disorders, amnesia, aggression, autism, vertigo, dementia, circadian rhythm disorders and gastric motility disorders e.g. IBS.

Within the context of the present invention, the terms describing the indications used herein are classified in the Diagnostic and Statistical Manual of Mental Disorders, 4th Edition, published by the American Psychiatric Association (DSM-IV) and/or the International Classification of Diseases, 10th Edition (ICD-10). The various subtypes of the disorders mentioned herein are contemplated as part of the present invention. Numbers in brackets after the listed diseases below refer to the classification code in DSM-IV.

Within the context of the present invention, the term “psychotic disorder” includes:—

Schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

Within the context of the present invention, the term “substance-related disorder” includes:—

Substance-related disorders including Substance Use Disorders such as Substance Dependence, Substance Craving and Substance Abuse; Substance-induced Disorders such as Substance Intoxication, Substance Withdrawal, Substance-induced Delirium, Substance-Induced Persisting Dementia, Substance-Induced Persisting Amnestic Disorder, Substance-induced Psychotic Disorder, Substance-Induced Mood Disorder, Substance-induced Anxiety Disorder, Substance-induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-induced Psychotic Disorder, Cocaine-induced Mood Disorder, Cocaine-Induced Anxiety Disorder, Cocaine-induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-induced Psychotic Disorder, Hallucinogen-Induced Mood Disorder, Hallucinogen-induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-induced Mood Disorder, Sedative-, Hypnotic-, or Anxiolytic-Induced Anxiety Disorder Sedative-, Hypnotic-, or Anxiolytic-induced Sexual Dysfunction, Sedative-, Hypnotic-, or Anxiolytic-induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

In a further aspect therefore the present invention provides a method of treating a condition for which modulation (especially antagonism/inhibition) of dopamine receptors (especially dopamine D₃ receptors) is beneficial, which comprises administering to a mammal (e.g. human) in need thereof an effective amount of a compound of formula (I) or a pharmaceutically (i.e physiologically) acceptable salt thereof. Such conditions in particular include psychoses/psychotic conditions such as schizophrenia, and substance abuse and/or drug dependency. For example, the condition to be treated may be craving for abused substance and/or relapse to drug seeking and drug taking behaviour.

The invention also provides the use of a compound of formula (I) or a pharmaceutically acceptable salt thereof in the manufacture of a medicament for the treatment of a condition in a mammal for which modulation (especially antagonism/inhibition) of dopamine receptors (especially dopamine D₃ receptors) is beneficial.

The invention also provides a compound of formula (I) or a pharmaceutically acceptable salt thereof for use in the treatment of a condition in a mammal for which modulation (especially antagonism/inhibition) of dopamine receptors (especially dopamine D₃ receptors) is beneficial.

In one embodiment, D₃ antagonists according to the present invention are used in the treatment of psychoses such as schizophrenia or in the treatment of substance abuse and/or drug dependency.

Thus, a still further aspect the invention provides a method of treating a psychotic condition (e.g. schizophrenia) or substance abuse and/or drug dependency which comprises administering to a mammal (e.g. human) in need thereof an effective amount of a compound of formula (I) as herein defined or a pharmaceutically acceptable salt thereof.

Also provided is the use of a compound of formula (I) or a pharmaceutically acceptable salt thereof in the manufacture of a medicament for the treatment of a psychotic condition (e.g. schizophrenia) or substance abuse and/or drug dependency in a mammal.

Also provided is a compound of formula (I) or a pharmaceutically acceptable salt thereof for use in the treatment of a psychotic condition (e.g. schizophrenia) or substance abuse and/or drug dependency in a mammal.

Also provided is a compound of formula (I) or a pharmaceutically acceptable salt thereof for use as an active therapeutic substance in a mammal, e.g. for use in the treatment of any of the conditions described herein.

“Treatment” includes prophylaxis, where this is appropriate for the relevant condition(s).

For use in medicine, the compounds of the present invention are usually administered as a standard pharmaceutical composition. The present invention therefore provides in a further aspect a pharmaceutical composition comprising a compound of formula (I) or a pharmaceutically (i.e physiologically) acceptable salt thereof and a pharmaceutically (i.e physiologically) acceptable carrier. The pharmaceutical composition can be for use in the treatment of any of the conditions described herein.

The compounds of formula (I) may be administered by any convenient method, for example by oral, parenteral (e.g. intravenous), buccal, sublingual, nasal, rectal or transdermal administration and the pharmaceutical compositions adapted accordingly.

The compounds of formula (I) and their pharmaceutically acceptable salts which are active when given orally can be formulated as liquids or solids, for example syrups, suspensions or emulsions, tablets, capsules and lozenges.

A liquid formulation will generally consist of a suspension or solution of the compound or pharmaceutically acceptable salt in a suitable liquid carrier(s) for example an aqueous solvent such as water, ethanol or glycerine, or a non-aqueous solvent, such as polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and cellulose.

A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of the compound or pharmaceutically acceptable salt in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active substance in a pharmaceutically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a propellant which can be a compressed gas such as compressed air or an organic propellant such as a fluoro-chlorohydrocarbon. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

In one embodiment, the composition is in unit dose form such as a tablet, capsule or ampoule.

Each dosage unit for oral administration contains for example from 1 to 250 mg (and for parenteral administration contains for example from 0.1 to 25 mg) of a compound of the formula (I) or a pharmaceutically acceptable salt thereof calculated as the free base.

The pharmaceutically acceptable compounds of the invention will normally be administered in a daily dosage regimen (for an adult patient) of, for example, an oral dose of between 1 mg and 500 mg, for example between 10 mg and 400 mg, e.g. between 10 and 250 mg or an intravenous, subcutaneous, or intramuscular dose of between 0.1 mg and 100 mg, for example between 0.1 mg and 50 mg, e.g. between 1 and 25 mg of the compound of the formula (I) or a pharmaceutically acceptable salt thereof calculated as the free base, the compound being administered 1 to 4 times per day. Suitably the compounds will be administered for a period of continuous therapy, for example for a week or more.

### Biological Test Methods

Functional potency and intrinsic activity of compounds of this invention can be measured by the following GTPγS scintillation proximity assay (GTPγS-SPA). Cells used in the study are Chinese Hamster Ovary (CHO) Cells.

### Cell Line

### CHO_D2

### CHO_D3

Cell membranes are prepared as follows. Cell pellets are resuspended in 10 volumes of 50 mM HEPES, 1 mM EDTA pH 7.4, using KOH. On the day the following proteases are added to the buffer just prior to giving the homogenisation buffer.

10⁻⁶M Leupeptin (Sigma L2884)-5000× stock=5 mg/ml in buffer
25 ug/ml Bacitracin (Sigma B0125)-1000× stock=25 mg/ml in buffer
1 mM PMSF—1000× stock=17 mg/ml in 100% ethanol
2×10⁻⁶M Pepstain A—1000× stock=2 mM in 100% DMSO

The cells are homogenised by 2×15 second bursts in a 1 litre Glass Waring blender in a class two biohazard cabinet. The resulting suspension is spun at 500 g for 20 mins (Beckman T21 centrifuge: 1550 rpm). The supernatant is withdrawn with a 25 ml pipette, aliquotted into pre-chilled centrifuge tubes and spun at 48,000 g to pellet membrane fragments (Beckman T1270: 23,000 rpm for 30 mins). The final 48,000 g pellet is resuspended in Homogenisation Buffer, (4× the volume of the original cell pellet). The 48,000 g pellet is resuspended by vortexing for 5 seconds and homogenized in a dounce homogenizer 10-15 stokes. The prep is distributed into appropriate sized aliquots, (200-1000 ul), in polypropylene tubes and store at −80° C. Protein content in the membrane preparations is evaluated with the Bradford protein assay.

The final top concentration of test drug is 3 uM in the assay and 11 points serial dilution curves 1:4 in 100% DMSO are carried out using a Biomek FX. The test drug at 1% total assay volume (TAV) is added to a solid, white, 384 well assay plate. 50% TAV of precoupled (for 90 mins at 4° C.) membranes, 5 μg/well, and Wheatgerm Agglutinin Polystyrene Scintillation Proximity Assay beads (RPNQO260, Amersham), 0.25 mg/well, in 20 mM HEPES pH 7.4, 100 mM NaCl, 10 mM MgCl₂, 60 μg/ml saponin and 30 μM GDP is added. The third addition was a 20% TAV addition of either buffer, (agonist format) or EC₈₀ final assay concentration of agonist, Quinelorane, prepared in assay buffer (antagonist format). The assay was started by the addition of 29% TAV of GTPγ[³⁵S] 0.38 nM final (37 MBq/ml, 1160 Ci/mmol, Amersham). After all additions assay plates are spun down for 1 min at 1,000 rpm. Assay plates are counted on a Viewlux, 613/55 filter, for 5 min., between 2-6 hours after the final addition.

The effect of the test drug over the basal generates EC₅₀ value by an iterative least squares curve fitting programme, expressed in the table as pEC₅₀ (i.e. -logEC₅₀). The ratio between the maximal effect of the test drug and the maximal effect of full agonist, Quinelorane, generates the Intrinsic Activity (IA) value (i.e. IA=1 full agonist, IA<1 partial agonist). fpki values of test drug are calculated from the IC₅₀ generated by “antagonist format” experiment, using Cheng & Prusoff equation: fKi=IC₅₀/1+([A]/EC₅₀) where: [A] is the concentration of the agonist 5-HT in the assay and EC₅₀ is the 5-HT EC₅₀ value obtained in the same experiment. fpKi is defined as -logfKi.

The compounds of the invention listed above have pKi values within the range of 7.5-9.5 at the dopamine D3 receptor. pKi results are only estimated to be accurate to about ±0.3-0.5.

The compounds of the invention listed above have a selectivity over D2 greater than 30.

### Human Histamine H1 Receptor Activity

Activity at the human Histamine H1 receptor can be measured using the general culture and assay conditions described in, for example, Smart et al, British Journal of Pharmacology (1999) 128, 1-3.

### EXAMPLES

The invention is further illustrated by the following non-limiting examples.

### Preparation 1: 3-(2-Chloroethyl)-7-(5-methyl-3-isoxazolyl)-2,3,4,5-tetrahydro-1H-3-benzazepine

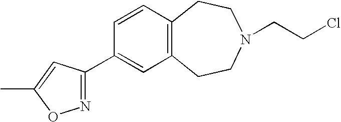

To a solution of 7-(5-methyl-3-isoxazolyl)-2,3,4,5-tetrahydro-1H-3-benzazepine (WO0240471A2) (0.21 g, 0.92 mol) in a mixture of dichloroethane (2 mL) and acetonitrile (2 mL) at room temperature was added chloroacetaldehyde (0.23 mL) followed by sodium triacetoxyborohydride (0.39 g). The reaction mixture was stirred at room temperature for 1 h. Solvent was removed in vacuo and the residue was dissolved in water (5 mL) and CH₂Cl₂ (5 mL). The mixture was extracted with CH₂Cl₂ (3×5 mL) and the organic layer was dried over Na₂SO₄. Filtration and evaporation gave the crude product which was purified by flash chromatography (silica gel/ethylacetate:cyclohexanes 2:8) to give 0.11 g of the title compound (41% yield).

NMR (¹H, CDCl₃): δ 7.5 (m, 1H), 7.45 (m, 1H), 7.15 (m, 1H), 6.2 (s, 1H), 3.60-3.55 (m, 2H), 2.95-2.85 (m, 6H), 2.75-2.70 (m, 4H), 2.45 (s, 3H).

### Example 1

### 7-(5-Methyl-3-isoxazolyl)-3-(2-{[4-methyl-1,3-oxazol-5yl)-4H-1,2,4-triazol-3-yl]thio}ethyl)-2,3,4,5-tetrahydro-1H-3-benzazepine hydrochloride

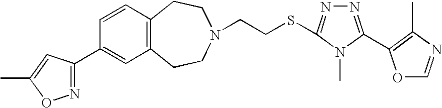

To a solution of 3-(2-chloroethyl)-7-(5-methyl-3-isoxazolyl)-2,3,4,5-tetrahydro-1H-3-benzazepine (0.03 g, 0.10 mmol) in dry DMF (0.5 mL), 4-methyl-5-(4-methyl-1,3-oxazol-5-yl)-4H-1,2,4-triazole-3-thiol (WO0240471A2) (0.10 mmol) was added followed by LiOH (0.14 mmol) and Nal (0.10 mmol). The reaction mixture was stirred at 90° C. for 16 h. Solvent was removed in vacuo and the residue was dissolved in water (5 mL) and CH₂Cl₂ (5 mL). The mixture was extracted with CH₂Cl₂ (3×5 mL) and the organic layer was dried over Na₂SO₄. Filtration and evaporation gave the crude product which was purified by flash chromatography (silica gel/CH₂Cl₂:MeOH 9:1) to give the free base of the title compound. To a solution of this material in CH₂Cl₂ (0.2 mL) was added 0.14 mmol of HCl (1M in Et₂O), the solvent evaporated in vacuo and the material thus obtained triturated with Et₂O to give 34 mg of the title compound as a white slightly hygroscopic solid (70% yield).

NMR (¹H, MeOD): δ 8.4 (s, 1H), 7.75 (d, 1H), 7.7 (dd, 1H), 7.41 (d, 1H), 6.6 (s, 1H), 4.0-3.84 (m, 2H), 3.80 (s, 3H), 3.78-3.70 (m, 4H), 3.52-3.18 (m, 6H) 2.51 (s, 3H), 2.47 (s, 3H). MS (m/z): 451.2 [MH]⁺.

### Example 2

### 7-(5-Methyl-3-isoxazolyl)-3-(2-{[4-methyl-5-(tetrahydro-2H-pyran-4-yl)-4H-1,2,4-triazol-3-yl]thio}ethyl)-2,3,4,5-tetrahydro-1H-3-benzazepine hydrochloride

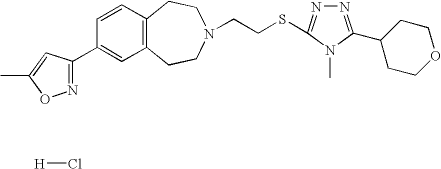

The title compound was prepared in analogy to the method described in Example 1 in 27 mg yield as a white slightly hygroscopic solid (61% yield) from 4-methyl-5-(tetrahydro-2H-pyran-4-yl)-4H-1,2,4-triazole-3-thiol (20 mg).

NMR (¹H, MeOD): δ 7.75 (d, 1H), 7.7 (dd, 1H), 7.41 (d, 1H), 6.6 (s, 1H), 3.77 (s, 3H), 3.4 (m, 1H), 4.1 (m, 2H), 3.4 (m, 2H), 4.0-3.2 (m, 12H), 2.5 (s, 3H), 1.99 (m, 4H). MS (m/z): 454.2 [MH]⁺

### Example 3

### 7-(5-Methyl-3-isoxazolyl)-3-(2-{[4-methyl-5-(2-methyl-5-quinolinyl)-4H-1,2,4-triazol-3-yl]thio}ethyl)-2,3,4,5-tetrahydro-1H-3-benzazepine hydrochloride

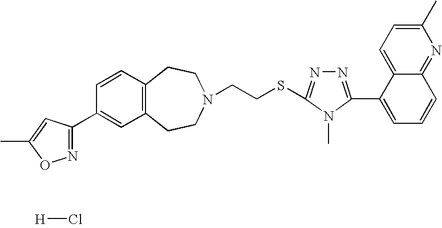

The title compound was prepared in analogy to the method described in Example 1 in 30 mg yield as a white slightly hygroscopic solid (61% yield) from 4-methyl-5-(2-methyl-5-quinolinyl)-4H-1,2,4-triazole-3-thiol (27 mg).

NMR (¹H, MeOD): δ 8.89 (d, 1H), 8.39 (d, 1H), 8.26 (t, 1H), 8.09 (d, 1H), 7.94 (d, 1H), 7.75 (d, 1H), 7.7 (dd, 1H), 7.41 (d, 1H), 6.6 (s, 1H), 4.0 (m, 2H), 3.66-3.23 (m, 10H), 3.81 (s, 3H), 3.01 (s, 3H), 2.5 (s, 3H). MS (m/z): 511.2 [MH]⁺.

### Example 4

### 7-(5-Methyl-3-isoxazolyl)-3-(2-{[4-methyl-5-(2-methyl-6-quinolinyl)-4H-1,2,4-triazol-3-yl]thio}ethyl)-2,3,4,5-tetrahydro-1H-3-benzazepine hydrochloride

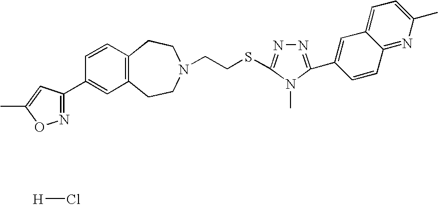

The title compound was prepared in analogy to the method described in Example 1 in 9 mg yield as a white slightly hygroscopic solid (18% yield) from 4-methyl-5-(2-methyl-6-quinolinyl)-4H-1,2,4-triazole-3-thiol (27 mg).

NMR (¹H, MeOD): δ 8.89 (d, 1H), 8.52, (d, 1H), 8.26 (dd, 1H), 8.2 (d, 1H), 7.87 (d, 1H), 7.63 (d, 1H), 7.57 (dd, 1H), 7.3 (d, 1H), 6.46 (s, 1H), 3.65, 3.73, 3.9-3.23 (s, s, bm, 15H), 3.73 (s, 3H), 3.65 (s, 3H), 3.01 (s, 3H), 2.5 (s, 3H). MS (m/z): 511.2 [MH]⁺.

### Synthetic Routes to Examples 5, 6 and 7:

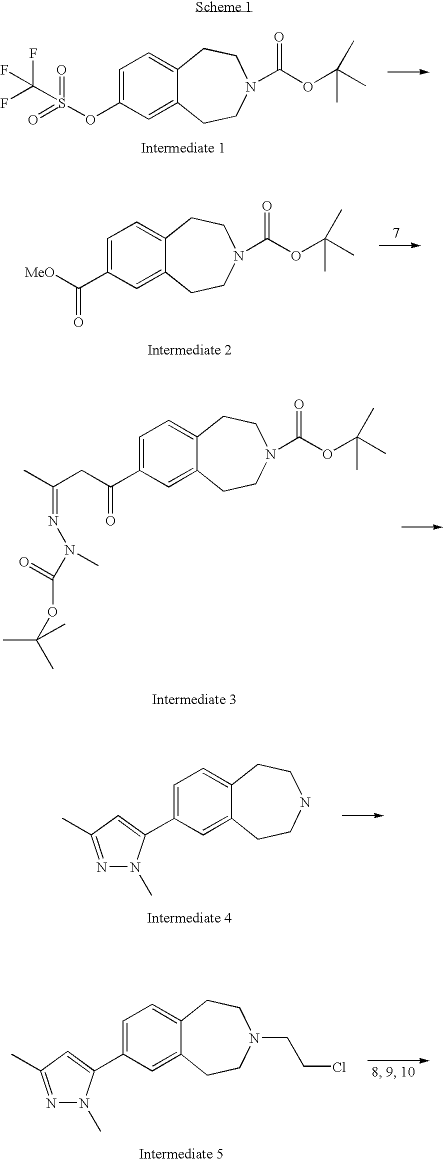

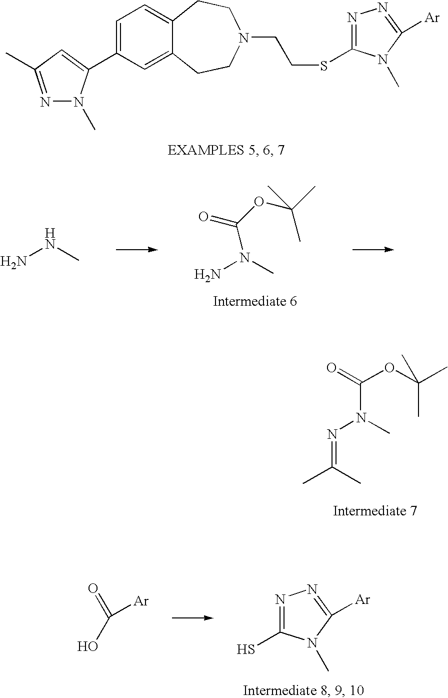

### Preparation 2: 3-(1,1-Dimethylethyl)-7-methyl-1,2,4,5-tetrahydro-3H-3-benzazepine-3,7-dicarboxylate (Intermediate 2

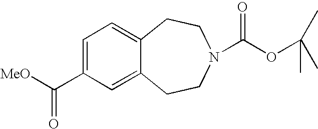

1,1-dimethylethyl-7-{[(trifluoromethyl)sulfonyl]oxy}-1,2,4,5-tetrahydro-3H-3-benzazepine-3-carboxylate (30 g) (WO/200240471), palladium (II) acetate (0.51 g) and 1,1′-bis(diphenylphosphino)ferrocene (1.25 g) were dissolved in anhydrous dimethylformamide (75 ml) and methanol (68 ml) under a nitrogen atmosphere, followed by addition of triethylamine (22.74 ml). The solution was purged with carbon monoxide for 15 min and stirred in a round-bottom flask equipped with a reservoir filled with carbon monoxide, at 70° C. for 18 h. The reaction mixture was allowed to reach room temperature, then dichloromethane (300 ml) and water (300 ml) were added. The organic phase was separated, dried with sodium sulphate and evaporated under reduced pressure. The crude product was purified by chromatography on silica gel with 90% cyclohexane-ethyl acetate elution to give the title compound (15 g) as an orange oil.

¹H-NMR (CDCl₃) δ: 7.79 (m, 2H), 7.18 (m, 1H), 3.89 (s, 3H), 3.57 (m, 4H), 2.95 (m, 4H), 1.48 (s, 9H).

### Preparation 3: 1,1-Dimethylethyl-7-{3-[{[(1,1-dimethylethyl)oxy]carbonyl}(methyl)-hydrazono]butanoyl}-1,2,4,5-tetrahydro-3H-3-benzazepine-3-carboxylate (Intermediate 3)

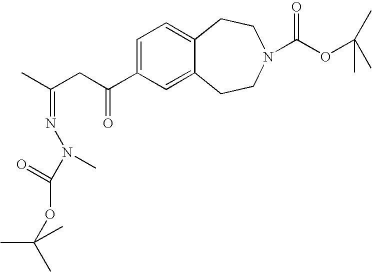

To a stirred solution of 1,1-dimethylethyl-1-methyl-2-(1-methylethylidene)-hydrazinecarboxylate (18.2 g) (Preparation 7) in tetrahydrofuran (80 ml), at 0° C., under a nitrogen atmosphere, lithium bis(trimethylsilyl)amide (115 ml, 1M/tetrahydrofuran) was added over 0.5 h keeping the temperature below 5° C. After stirring for an additional hour, the reaction mixture was added via cannula to a stirred solution of 3-(1,1-dimethylethyl)-7-methyl-1,2,4,5-tetrahydro-3H-3-benzazepine-3,7-dicarboxylate (10 g) (Preparation 2) in anhydrous tetrahydrofuran (70 ml), at 0° C., under a nitrogen atmosphere. Stirring was continued for 2 h after which time water (300 ml) was added and the reaction mixture was extracted with ethyl acetate (800 ml). The organic phase was washed with brine (400 ml), dried with sodium sulphate and concentrated under reduced pressure. The crude product was purified by chromatography on silica gel with 70% cyclohexane-ethyl acetate to give the title compound (12 g) as a white solid.

¹H-NMR (DMSO-d₆) δ: 11.65 (s, 1H), 7.67 (d, 1H), 7.64 (dd, 1H), 7.23 (d, 1H), 5.90 (s, 1H), 3.47 (m, 4H), 3.10 (s, 3H), 2.90 (bm, 4H), 1.98 (s, 3H), 1.41 (s, 18H).

### Preparation 4: 7-(1,3-Dimethyl-1H-pyrazol-5-yl)-2,3,4,5-tetrahydro-1H-3-benzazepine (Intermediate 4)

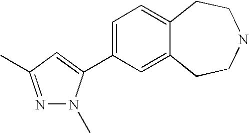

A solution of 1,1-dimethylethyl-7-{3-[{[(1,1dimethylethyl)oxy]carbonyl}(methyl)-hydrazono]butanoyl}-1,2,4,5-tetrahydro-3H-3-benzazepine-3-carboxylate (0.5 g) (Preparation 3) in dichloromethane (5 ml) was added dropwise to trifluoroacetic acid (10 ml) under vigorous stirring. After 1 h the reaction mixture was concentrated in vacuo and sodium hydroxide (1N) was added until pH ˜12, then the mixture was extracted twice with dichloromethane. The organic phase was dried with sodium sulphate and evaporated to give the title compound (0.26 g).

¹H-NMR (DMSO-d₆) δ: 7.2-7.1 (m, 3H), 6.06 (s, 1H), 3.73 (s, 3H), 2.9-2.7 (m, 8H), 2.5 (3H).

### Preparation 5: 3-(2-Chloroethyl)-7-(1,3-dimethyl-1H-pyrazol-5-yl)-2,3,4,5-tetrahydro-1H-3-benzazepine (Intermediate 5)

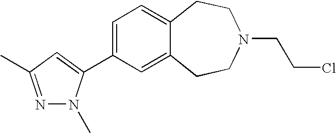

To a stirred solution of 7-(1,3-dimethyl-1H-pyrazol-5-yl)-2,3,4,5-tetrahydro-1H-3-benzazepine (0.6 g) (preparation 4) in 1,2-dichloroethane (10 ml), 3-chloropropanal (0.64 ml, 50 wt. % solution in water) and sodium triacetoxyborohydride (1.06 g) were subsequently added. After stirring for 1 h, the reaction was quenched with concentrated aqueous sodium hydrogencarbonate and extracted with dichloromethane. The organic phase was dried with sodium sulphate and after evaporation the crude product was purified by chromatography on silica gel with 80-20% cyclohexane-ethyl acetate elution to give the title compound (0.4 g) as a pale yellow solid.

### Preparation 6: 1,1-Dimethylethyl-1-methylhydrazine carboxylate (Intermediate 6)

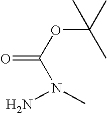

To a solution of methylhydrazine (100 g) in anhydrous tetrahydrofuran (1.8 L), cooled at 5° C. and stirred with a mechanic equipment, a solution of di-tert-butyl dicarbonate (498 g) in anhydrous tetrahydrofuran (600 ml) was added keeping this temperature for 0.5 h. Then water (500 ml) was added, followed by ethyl acetate (2 L). The organic phase was washed with water (2 L), brine (1.6 L) and dried with sodium sulphate, to give after evaporation under reduced pressure the title compound (230 g) as a white solid.

¹H-NMR (CDCl₃) δ: 3.84 (broad, 2H), 3.02 (s, 3H), 1.42 (s, 9H)

### Preparation 7: 1,1-Dimethylethyl-1-methyl-2-(1-methylethylidene) hydrazinecarboxylate (Intermediate 7)

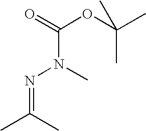

To a stirred solution of 1,1-dimethylethyl-1-methylhydrazine carboxylate (179 g) (Preparation 6) in diethyl ether (2 L), at room temperature, acetone (126 ml), glacial acid acetic (7.7 ml) and sodium acetate (1.27 g) were added. After stirring over night, the reaction mixture was quenched with water, the organic phase was dried with sodium sulphate and the solvent evaporated to give the title compound (182.38 g) as a colourless oil.

¹H-NMR (CDCl₃) δ: 3.01 (s, 3H), 2.01 (s, 3H), 1.83 (s, 3H), 1.42 (s, 9H).

### Preparation 8: 4-methyl-5-(2-methyl-5-quinolinyl)-4H-1,2,4-triazole-3-thiol (Intermediate 8)

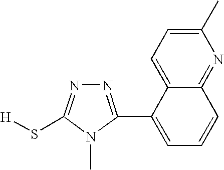

Hydroxybenzotriazole (7.8 g), 1-(3-dimethylaminopropyl)-3-ethylcarbodiimide hydrochloride (11 g) and triethylamine were successively added to a stirred solution of 2-methyl-5-quinolinecarboxylic acid (10 g) and 4-methyl-3-thiosemicarbazide (6.1 g) in dimethylformamide (200 ml), at 0° C. Following the addiction the reaction mixture was allowed to reach room temperature, the stirred continued over night and then the solvent was evaporated under reduced pressure. The residue was treated with an aqueous sodium hydroxide solution (500 ml, 0.5N) and the mixture was stirred at 80° C. for 3 h, after which time the mixture was cooled to room temperature and the pH adjusted to pH 6 using a aqueous hydrochloridric acid solution (2M) and the resulting precipitate was filtered and dried in vacuo to give the title compound (11 g) as an off-white solid.

¹H-NMR (DMSO-d₆) δ: 14 (broad, 1H), 8.17 (dd, 1H), 8.15 (dd, 1H), 7.89 (m, 1H), 7.85 (dd, 1H), 7.52 (dd, 1H), 3.32 (s, 3H), 2.70 (s, 3H).

### Preparation 9: 4-Methyl-5-(5-methyl-2-pyrazinyl)-4H-1,2,4-triazole-3-thiol (Intermediate 9)

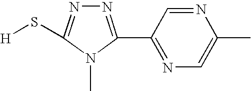

Hydroxybenzotriazole (1.08 g) and 1-(3-dimethylaminopropyl)-3-ethylcarbodiimide hydrochloride (1.53 g) were successively added to a stirred solution of 5-methyl-2-pyrazinecarboxylic acid (1 g) and 4-methyl-3-thiosemicarbazide (0.84 g) in dimethylformamide (20 ml), at 0° C. Following the addiction the reaction mixture was allowed to reach room temperature, the stirred continued over night and then the solvent was evaporated under reduced pressure. The residue was treated with an aqueous sodium hydroxide solution (10 ml, 0.5N) and the mixture was stirred at 80° C. for 3 h, after which time the mixture was cooled to room temperature and the pH adjusted to pH 6 using a aqueous hydrochloridric acid solution (2M) and the resulting precipitate was filtered and dried in vacuo to give the title compound (1.30 g) as an off-white solid.

¹H-NMR (DMSO-d₆) δ: 14 (bs, 1H), 8.94 (s, 1H), 8.60 (s, 1H), 3.68 (s, 3H), 2.50 (s, 3H).

### Preparation 10: 5-(3,4-difluorophenyl)-4-methyl-4H-1,2,4-triazole-3-thiol (Intermediate 10)

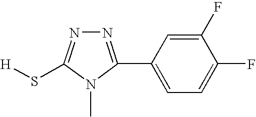

Hydroxybenzotriazole (4.22 g) and 1-(3-dimethylaminopropyl)-3-ethylcarbodiimide hydrochloride (5.99 g) were successively added to a stirred solution of 3,4-difluorobenzoic acid (4.49 g) and 4-methyl-3-thiosemicarbazide (3.28 g) in dimethylformamide (80 ml), at 0° C. Following the addiction the reaction mixture was allowed to reach room temperature, the stirred continued over night and then the solvent was evaporated under reduced pressure. The residue was treated with an aqueous sodium hydroxide solution (250 ml, 0.5N) and the mixture was stirred at 80° C. for 3 h, after which time the mixture was cooled to room temperature and the pH adjusted to pH 6 using a aqueous hydrochloridric acid solution (2M) and the resulting precipitate was filtered and dried in vacuo to give the title compound (4.1 g) as an off-white solid.

¹H-NMR (DMSO-d₆) δ: 13.95 (bs, 1H), 7.90 (m, 1H), 7.65 (m, 2H), 3.50 (s, 3H).

### Example 5

### 7-(1,3-Dimethyl-1H-pyrazol-5-yl)-3-(2-{[4-methyl-5-(2-methyl-5-quinolinyl)-4H-1,2,4-triazol-3-yl]-thio}ethyl)-2,3,4,5-tetrahydro-1H-3-benzazepine hydrochloride

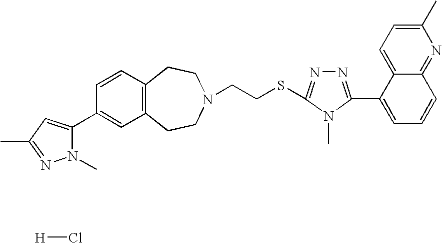

To a stirred solution of 3-(2-chloroethyl)-7-(1,3-dimethyl-1H-pyrazol-5-yl)-2,3,4,5-tetrahydro-1H-3-benzazepine (0.13 g) (Preparation 5) and 4-methyl-5-(2-methyl-5-quinolinyl)-2,4-dihydro-3H-1,2,4-triazole-3-thione (0.11 g) (Preparation 8) in dimethylformamide (2 ml), at room temperature, N,N-diisopropylethylamine (0.09 ml) and sodium iodide (0.06 g) were subsequently added. The reaction mixture was warmed to 70° C. and stirring continued for 3 h after which time the mixture was allowed to reach room temperature and the solvent evaporated under reduced pressure. The residue was treated with water (10 ml) and extracted with ethyl acetate (20 ml). The organic phase was dried with sodium sulphate, evaporated under reduced pressure and the crude product was purified by chromatography on silica gel with 100-95% dichloromethane-methanol elution to give 7-(1,3-dimethyl-1H-pyrazol-5-yl)-3-(1-methyl-3-{[4-methyl-5-(2-methyl-5-quinolinyl)-4H-1,2,4-triazol-3-yl]thio}propyl)-2,3,4,5-tetrahydro-1H-3-benzazepine (0.040 g) as a pale yellow solid. This product was dissolved in dichloromethane (2 ml), and hydrochloridric acid was added dropwise (0.072 ml, 1M/ether), at room temperature. Following solvent evaporation gave the title compound (0.042 g) as a yellow solid.

¹H-NMR (DMSO-d₆) δ: 10.82 (bs, 1H), 8.26 (d, 1H), 8.19 (d, 1H), 7.92 (t, 1H), 7.80 (d, 1H), 7.56 (d, 1H), 7.37 (m, 3H), 6.16 (m, 1H), 3.90-3.80 (bm, 2H), 3.77 (s, 3H), 3.70 (m, 2H), 3.65 (m, 2H), 3.46 (s, 3H), 3.50-3.10 (bm, 6H), 2.74 (s, 3H), 2.17 (s, 3H).

### Example 6

### 7-(1,3-Dimethyl-1H-pyrazol-5-yl)-3-(2-{[4-methyl-5-(5-methyl-2-pyrazinyl)-4H-1,2,4-triazol-3-yl]thio}ethyl)-2,3,4,5-tetrahydro-1H-3-benzazepine hydrochloride

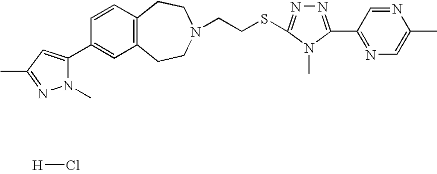

To a stirred solution of 3-(2-chloroethyl)-7-(1,3-dimethyl-1H-pyrazol-5-yl)-2,3,4,5-tetrahydro-1H-3-benzazepine (0.13 g) (Preparation 5) and 4-methyl-5-(5-methyl-2-pyrazinyl)-2,4-dihydro-3H-1,2,4-triazole-3-thione (0.09 g) (Preparation 9) in dimethylformamide (2 ml), at room temperature, N,N-diisopropylethylamine (0.09 ml) and sodium iodide (0.06 g) were subsequently added. The reaction mixture was warmed to 70° C. and stirring continued for 3 h after which time the mixture was allowed to reach room temperature and the solvent evaporated under reduced pressure. The residue was treated with water (10 ml) and extracted with ethyl acetate (20 ml). The organic phase was dried with sodium sulphate, evaporated under reduced pressure and the crude product was purified by chromatography on silica gel with 100-95% dichloromethane-methanol elution to give 7-(1,3-dimethyl-1H-pyrazol-5-yl)-3-(2-{[4-methyl-5-(5-methyl-2-pyrazinyl)-4H-1,2,4-triazol-3-yl]thio}ethyl)-2,3,4,5-tetrahydro-1H-3-benzazepine (0.036 g) as a yellow solid. This product was dissolved in dichloromethane (2 ml), and hydrochloridric acid was added dropwise (0.076 ml, 1M/ether), at room temperature. Following solvent evaporation gave the title compound (0.038 g) as a yellow solid.

¹H-NMR (DMSO-d₆) δ: 10.65 (bs, 1H), 9.18 (d, 1H), 8.71 (d, 1H), 7.35 (m, 3H), 6.15 (s, 1H), 3.91 (s, 3H), 3.80-3.70 (bm, 2H), 3.76 (s, 3H), 3.66 (m, 2H), 3.58 (m, 2H), 3.40-3.30 (bm, 2H), 3.15 (bm, 4H), 2.61 (s, 3H), 2.16 (s, 3H).

### Example 7

### 3-(2-{[5-(3,4-Difluorophenyl)-4-methyl-4H-1,2,4-triazol-3-yl]thio}ethyl)-7-(1,3-dimethyl-1H-pyrazol-5-yl)-2,3,4,5-tetrahydro-1H-3-benzazepine hydrochloride

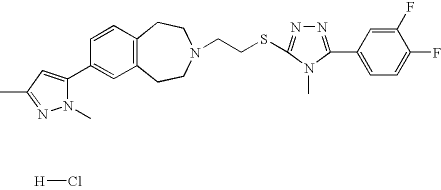

To a stirred solution of 3-(2-chloroethyl)-7-(1,3-dimethyl-1H-pyrazol-5-yl)-2,3,4,5-tetrahydro-1H-3-benzazepine (0.13 g) (Preparation 5) and 5-(3,4-difluorophenyl)-4-methyl-2,4-dihydro-3H-1,2,4-triazole-3-thione (0.1 g) (Preparation 10) in dimethylformamide (2 ml), at room temperature, N,N-diisopropylethylamine (0.09 ml) and sodium iodide (0.06 g) were subsequently added. The reaction mixture was warmed to 70° C. and stirring continued for 3 h after which time the mixture was allowed to reach room temperature and the solvent evaporated under reduced pressure. The residue was treated with water (10 ml) and extracted with ethyl acetate (20 ml). The organic phase was dried with sodium sulphate, evaporated under reduced pressure and the crude product was purified by chromatography on silica gel with 100-95% dichloromethane-methanol elution to give 3-(2-{[5-(3,4-difluorophenyl)-4-methyl-4H-1,2,4-triazol-3-yl]thio}ethyl)-7-(1,3-dimethyl-1H-pyrazol-5-yl)-2,3,4,5-tetrahydro-1H-3-benzazepine (0.05 g) as a pale white solid. This product was dissolved in dichloromethane (2 ml), and hydrochloridric acid was added dropwise (0.10 ml, 1M/ether), at room temperature. Following solvent evaporation gave the title compound (0.053 g) as a yellow solid.

¹H-NMR (DMSO-d₆) δ: 10.65 (bs, 1H), 7.85 (ddd, 1H), 7.63 (m, 1H), 7.35 (m, 3H), 6.15 (s, 1H), 3.80-3.70 (bm, 2H), 3.77 (s, 3H), 3.65 (s, 3H), 3.63 (m, 2H), 3.58 (m, 2H), 3.40-3.30 (bm, 2H), 3.15 (bm, 4H), 2.17 (s, 3H).

### Synthesis of Array

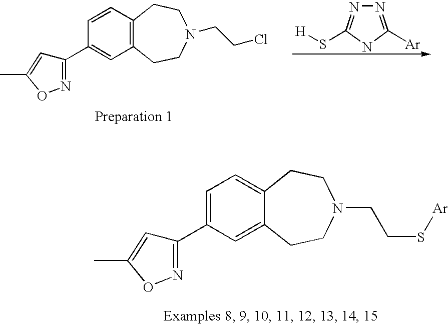

To a solution of the thioheteroaryl (0.061 mmol) (prepared in analogy as reported in WO/200240471) in dry acetonitrile (1 ml) 2-tert-butylimino-2-diethylamino-1,3-dimethyl-perhydro-1,3,2-diaza-phosphorine on polystyrene (41 mg, 2.2 mmol/g) was added and the resulting mixture was shaken for 30 minutes at room temperature then 3-(2-chloroethyl)-7-(5-methyl-3-isoxazolyl)-2,3,4,5-tetrahydro-1H-3-benzazepine (Preparation 1) (18 mg) was added and the resulting mixture was shaken at 70° C. for three hours. After cooling the resin was filtered off, washed with methanol (2 ml) and then the solvent was removed under reduced pressure. Purifications were carried out using mass directed HPLC using a Waters XTerra Prep MS C18 10 μm, 30×150 mm column using the following conditions

| | Time | Flow | % A | % B |
|---|---|---|---|---|
| Prerun | 0 | 40 ml/min | 99 | 1 |
| | 1 | 40 ml/min | 99 | 1 |
| Run | 0 | 40 ml/min | 99 | 1 |
| | 10 | 40 ml/min | 75 | 25 |
| | 14.5 | 40 ml/min | 10 | 90 |
| | 15 | 40 ml/min | 0 | 100 |
| Postrun | 0 | 40 ml/min | 0 | 100 |
| | 0.2 | 45 ml/min | 0 | 100 |
| | 1.5 | 45 ml/min | 0 | 100 |
| | 2 | 40 ml/min | 0 | 100 |

A = H2O + 0.1% formic acid
B = ACN + 0.1% formic acid

Then solvent was removed under reduced pressure to give title compounds as formate salts.

### HPLC:

### Analytical

### Column: X Terra MS C18 5 mm, 50×4.6 mm

Mobile phase: A: H20+0.2% HCOOH; B: CH₃CN+0.2% HCOOH
Gradient: 10% (B) for 1 min, from 10% (B) to 95% (B) in 12 min, 95% (B) for 3 min
Flow rate: 1 ml/min
UV wavelength range: 200-400 nm
Mass range: 100-900 amu

### Ionization: ES+

| Ex. | | Retention | Analytical |
|---|---|---|---|
| No | Structure and Name | Time | data |
| 8 | 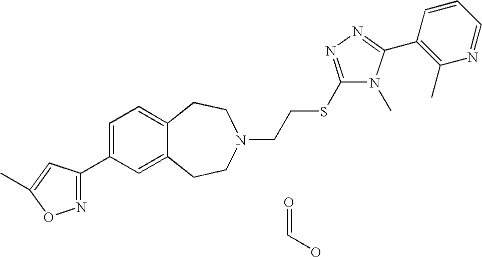 | 5.2 | MS (m/z):461 [MH]+ |
| | 7-(5-methyl-3-isoxazolyl)-3-(2-{[4-methyl-5-(2-methyl-3- | | |
| | pyridinyl)-4H-1,2,4-triazol-3-yl]thio}ethyl)-2,3,4,5- | | |
| | tetrahydro-1H-3-benzazepine formate | | |
| 9 | 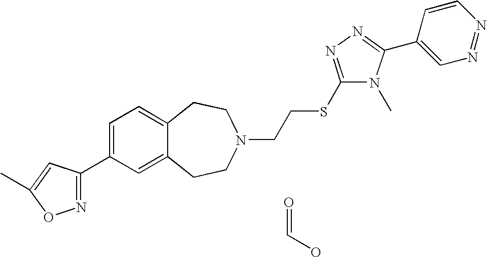 | 5.2 | MS (m/z):448 [MH]+ |
| | 7-(5-methyl-3-isoxazolyl)-3-(2-{[4-methyl-5-(4- | | |
| | pyridazinyl)-4H-1,2,4-triazol-3-yl]thio}ethyl)-2,3,4,5- | | |
| | tetrahydro-1H-3-benzazepine formate | | |
| 10 | 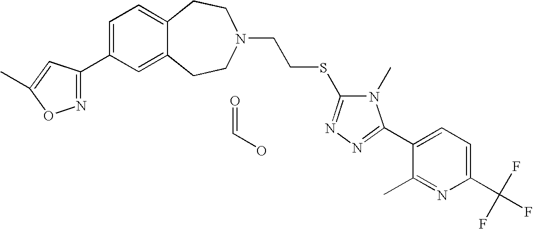 | 7.0 | MS (m/z):529 [MH]+ |
| | 7-(5-methyl-3-isoxazolyl)-3-[2-({4-methyl-5-[2-methyl-6- | | |
| | (trifluoromethyl)-3-pyridinyl]-4H-1,2,4-triazol-3- | | |
| | yl}thio)ethyl]-2,3,4,5-tetrahydro-1H-3-benzazepine | | |
| | formate | | |
| 11 | 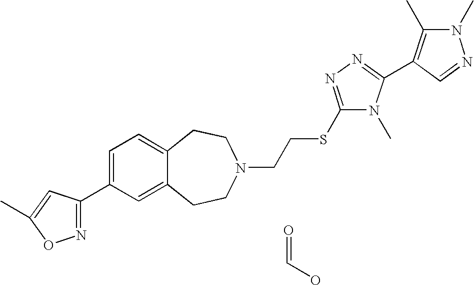 | 5.8 | MS (m/z):464 [MH]+ |
| | 3-(2-{[5-(1,5-dimethyl-1H-pyrazol-4-yl)-4-methyl-4H- | | |
| | 1,2,4-triazol-3-yl]thio}ethyl)-7-(5-methyl-3-isoxazolyl)- | | |
| | 2,3,4,5-tetrahydro-1H-3-benzazepine formate | | |
| 12 | 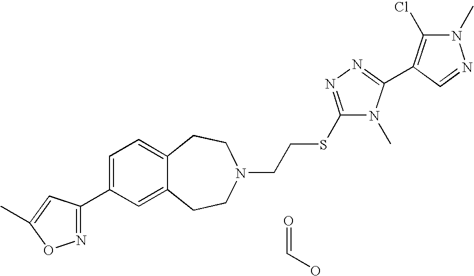 | 6.0 | MS (m/z):485 [MH]+ |
| | 3-(2-{[5-(5-chloro-1-methyl-1H-pyrazol-4-yl)-4-methyl- | | |
| | 4H-1,2,4-triazol-3-yl]thio}ethyl)-7-(5-methyl-3- | | |
| | isoxazolyl)-2,3,4,5-tetrahydro-1H-3-benzazepine | | |
| | formate | | |
| 13 | 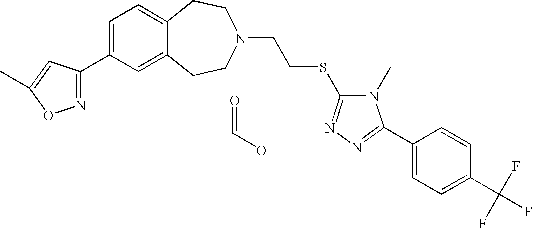 | 7.5 | MS (m/z):514 [MH]+ |
| | 7-(5-methyl-3-isoxazolyl)-3-[2-({4-methyl-5-[4- | | |
| | (trifluoromethyl)phenyl]-4H-1,2,4-triazol-3-yl}thio)ethyl]- | | |
| | 2,3,4,5-tetrahydro-1H-3-benzazepine formate | | |
| 14 | 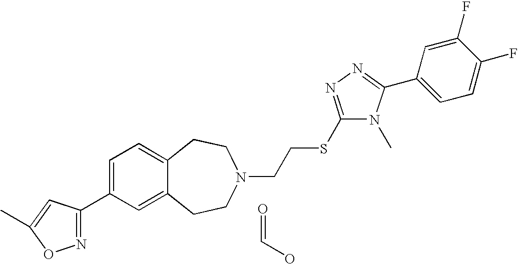 | 6.9 | MS (m/z):482 [MH]+ |
| | 3-(2-{[5-(3,4-difIuorophenyl)-4-methyl-4H-1,2,4-triazol- | | |
| | 3-yl]thio}ethyl)-7-(5-methyl-3-isoxazolyl)-2,3,4,5- | | |
| | tetrahydro-1H-3-benzazepine formate | | |
| 15 | 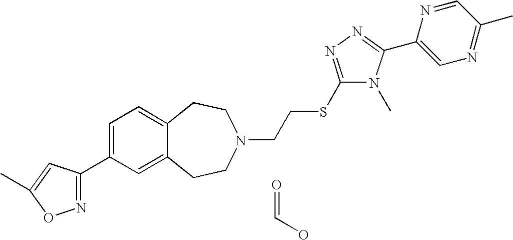 | 6.1 | MS (m/z):462 [MH]+ |
| | 7-(5-methyl-3-isoxazolyl)-3-(2-{[4-methyl-5-(5-methyl-2- | | |
| | pyrazinyl)-4H-1,2,4-triazol-3-yl]thio}ethyl)-2,3,4,5- | | |
| | tetrahydro-1H-3-benzazepine formate | | |

1. A compound of formula (I) or a
pharmaceutically acceptable salt thereof: 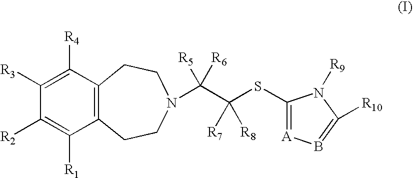
wherein
R₁ and R₄ are independently selected from the group consisting of hydrogen, fluoro, chloro, bromo, C₁₋₂alkyl, C₁alkoxy, haloC₁₋₂alkyl, haloC₁alkoxy, hydroxy, cyano and nitro;
R₂ and R₃ are independently selected from the group consisting of:
hydrogen, halogen, hydroxy, cyano, nitro, C₁₋₄alkyl, haloC₁₋₄alkyl, C₃₋₆cycloalkyl, C₁₋₄alkoxy, haloC₁₋₄alkoxy, C₁₋₄alkoxyC₁₋₄alkoxy, C₁₋₄alkylthio, C₁₋₄alkoxyC₁₋₄alkyl, C₃₋₆cycloalkylC₁₋₄alkoxy, C₁₋₄alkanoyl, C₁₋₄alkoxycarbonyl, C₁₋₄alkoxycarbonylC₁₋₄alkyl, C₁₋₄alkylsulfonyl, C₁₋₄alkylsulfonyloxy, haloC₁₋₄alkylsulfonyl, haloC₁₋₄alkylsulfonyloxy, C₁₋₄alkylsulfonylC₁₋₄alkyl, C₁₋₄alkylsulfonamido, C₁₋₄alkylsulfonamidoC₁₋₄alkyl, heterocyclyl, aryl, arylC₁₋₄alkoxy, aryloxy, arylthio, arylmethyl, aroyl, aryloxymethyl, arylsulfonyl, aryl-NR′— (wherein R′ is hydrogen or C₁₋₄alkyl), arylsulfonyloxy, arylsulfonylC₁₋₄alkyl, arylsulfonamido, arylcarboxamido, arylsulfonamidoC₁₋₄alkyl, arylcarboxamidoC₁₋₄alkyl, aroylC₁₋₄alkyl, arylC₁₋₄alkanoyl, a group R₁₁CON(R₁₂)(CH₂)ᵣ, R₁₁R₁₂NCO(CH₂)ᵣ or R₁₁R₁₂NSO₂(CH₂)ᵣ (in which r is 0, 1, 2, 3 or 4, and each of R₁₁ and R₁₂ is independently hydrogen or C₁₋₄alkyl, or in the groups R₁₁CON(R₁₂)(CH₂)ᵣ, R₁₁R₁₂NCO(CH₂)ᵣ and R₁₁R₁₂NSO₂(CH₂)ᵣ, R₁₁CONR₁₂ or R₁₁R₁₂N together form a 4-, 5-, 6- or 7-membered azacyclic group optionally containing one additional O, N or S atom in the azacycle and having 3-8 carbon atoms (including the carbon atoms contained in any optional substituent(s) of the azacycle)); wherein in any group containing an aryl moiety, the aryl may be substituted by one, two or three groups selected from the group consisting of halogen, hydroxy, cyano, nitro, amino, C₁₋₄alkyl, haloC₁₋₄alkyl, C₁₋₄alkoxy, haloC₁₋₄alkoxy, C₁₋₄alkylenedioxy, C₁₋₄alkanoyl, C₁₋₄alkylsulfonyl, haloC₁₋₄alkylsulfonyl, C₁₋₄alkylamino, C₁₋₄dialkylamino, R₁₃R₁₄NCO (in which R₁₃ and R₁₄ are independently hydrogen or C₁₋₄alkyl, or R₁₃R₁₄N together form a 4-, 5-, 6- or 7-membered azacyclic group optionally containing one additional O, N or S atom in the azacycle and having 3-8 carbon atoms (including the carbon atoms contained in any optional substituent(s) of the azacycle));
A and B are independently N or CH;
R₅, R₆, R₇, R₈ and R₉ are independently hydrogen or C₁₋₄alkyl;
R₁₀ is a group of the formula (a) or (b):
—Z (a)
—(CR₁₅R₁₆)ₜZ (b)
wherein:
Z is C₁₋₄alkyl, haloC₁₋₄alkyl, C₃₋₆cycloalkyl, phenyl, heterocyclyl, a 5- or 6-membered heteroaromatic ring or a 8- to 11-membered bicyclic group, any of which is optionally substituted by 1, 2, 3 or 4 substituents selected from the group consisting of: halogen, hydroxy, oxo, cyano, nitro, C₁₋₄alkyl, C₁₋₄alkoxy, haloC₁₋₄alkyl, haloC₁₋₄alkoxy, C₁₋₄alkylenedioxy, C₁₋₄alkanoyl, C₁₋₄alkylsulfonyl, C₁₋₄alkylsulfonyloxy, haloC₁₋₄alkylsulfonyl, haloC₁₋₄alkylsulfonyloxy, C₁₋₄alkylsulfinyl, C₁₋₄alkylthio, R₁₇SO₂N(R₁₈)—, R₁₇R₁₈NSO₂—, R₁₇R₁₈N—, R₁₇R₁₈NCO—, R₁₇CONR₁₈— and a 5- or 6-membered heteroaromatic ring which is optionally substituted by one or two C₁₋₂alkyl, haloC₁₋₂alkyl or R₁₇R₁₈N— (wherein R₁₇ and R₁₈ are independently hydrogen or C₁₋₄alkyl, or R₁₇ and R₁₈ together form C₃₋₆ alkylene); and wherein substituents positioned ortho to one another may be linked to form a 5- or 6-membered ring; and
R₁₅ and R₁₆ are independently hydrogen or C₁₋₄alkyl and t is 1, 2, 3 or 4, or —(CR₁₅R₁₆)ₜ— forms a C₃₋₆cycloalkylene linker.

2. A compound as claimed in claim 1, wherein R₃ is hydrogen.

3. A compound as claimed in claim 1 or claim 2, wherein R₂ is C₁₋₄alkyl, haloC₁₋₄alkyl, halogen, C₁₋₄alkylsulfonyl (e.g. methylsulfonyl or ethylsulfonyl), haloC₁₋₄alkylsulfonyl (e.g. trifluoromethylsulfonyl), C₁₋₄alkylsulfonyloxy (e.g. methylsulfonyloxy), haloC₁₋₄alkylsulfonyloxy (e.g. trifluoromethylsulfonyloxy), R₁₁R₁₂NSO₂ (where each of R₁₁ and R₁₂ is independently hydrogen or C₁₋₄alkyl or R₁₁R₁₂N together form a 4-, 5-, 6- or 7-membered azacyclic group optionally containing one additional O, N or S atom in the azacycle and having 3-8 carbon atoms, e.g. a piperidin-1-ylsulfonyl, pyrrolidin-1-ylsulfonyl or 1,4-morpholin-4-ylsulfonyl), a 5- or 6-membered heteroaromatic or a heterocyclyl, each of which is optionally substituted by one or two substituents selected from: halogen, cyano, C₁₋₂alkyl (e.g. methyl or trifluoromethyl), C₁₋₂alkoxy (e.g. methoxy), C₁₋₂alkylenedioxy (e.g. methylenedioxy), C₁₋₃alkanoyl (e.g. acetyl), C₂alkanoylamino (e.g. acetylamino), haloC₁alkylsulfonyl (e.g. trifluoromethylsulfonyl) and methylsulfonyl.

4. A compound as claimed in claim 3, wherein R₂ is bromo, cyano, hydroxy, chloro, methoxy, tert-butyl, methylsulfonyl, ethylsulfonyl, N,N-dimethylaminosulfonyl, pyrrolidin-1-ylsulfonyl, 1,4-morpholin-4-ylsulfonyl, methylsulfonyloxy, pyrazolyl (eg pyrazol-5-yl), 1,3-dimethyl-pyrazol-5-yl, pyrazin-2-yl, 5-methyl-oxazol-2-yl or 5-methyl-isoxazol-3-yl.

5. A compound as claimed in claim 1, wherein both R₁ and R₄ are hydrogen.

6. A compound as claimed in claim 1, wherein A and B are both nitrogen.

7. A compound as claimed in claim 1, wherein R₅, R₆, R₇ and R₈ are all hydrogen.

8. A compound as claimed in claim 1, wherein R₉ is methyl.

9. A compound as claimed in claim 1, wherein R₁₀ is a group of formula (a).

10. A compound as claimed in claim 9, wherein in formula (a), Z is phenyl, fluorophenyl, or quinolinyl, each of which is unsubstituted or substituted by one or more substituents selected from: halogen, or cyano, C₁₋₂alkyl (e.g. methyl), haloC₁₋₂alkyl (e.g. trifluoromethyl), C₁₋₂alkoxy (e.g. methoxy), haloC₁₋₄alkoxy (e.g. trifluoromethoxy), C₁₋₂alkylenedioxy (e.g. methylenedioxy), C₂₋₃alkanoyl (e.g. acetyl), C₂alkanoylamino (e.g. acetylamino), methylsulfonyl, haloC₁alkylsulfonyl (e.g. trifluoromethylsulfonyl), C₁alkylsulfonyloxy (e.g. methylsulfonyloxy), C₁alkylaminosulfonyl (e.g. methylaminosulfonyl), C₁alkylsulfonylamino (e.g. methylsulfonylamino) and C₁alkylaminocarbonyl (e.g. methylaminocarbonyl).

11. A compound as claimed in claim 1 having a formula (IA) or a pharmaceutically acceptable salt thereof: 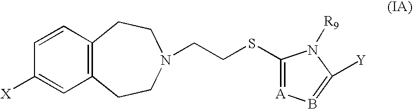
wherein:
A, B and R₉ are as defined in claim 1;
X is a 5- or 6-membered heteroaromatic ring optionally substituted by 1, 2 or 3 substituents selected from the group consisting of: halogen, cyano, C₁₋₂alkyl, fluoroC₁₋₂alkyl, C₁₋₂alkoxy, C₁₋₃alkanoyl, C₂alkanoylamino, fluoroC₁alkylsulfonyl and methylsulfonyl; and
Y is phenyl, heterocyclyl, a 5- or 6-membered heteroaromatic ring or a 8- to 11-membered bicyclic group, any of which is optionally substituted by 1, 2, 3 or 4 substituents selected from the group consisting of: halogen, cyano, C₁₋₂alkyl, haloC₁₋₂alkyl, C₁₋₂alkoxy, haloC₁₋₂alkoxy, C₁₋₂alkylenedioxy, C₂₋₃alkanoyl, C₂alkanoylamino, methylsulfonyl, haloC₁alkylsulfonyl, methylsulfonyloxy, methylaminosulfonyl, methylsulfonylamino and methylaminocarbonyl.

12. A compound as claimed in claim 1 having a formula (IB) or a pharmaceutically acceptable salt thereof: 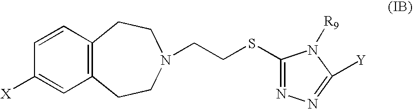
wherein
X is isoxazolyl or pyrazolyl ring optionally substituted by 1, 2 or 3 substituents selected from the group consisting of: halogen, cyano, C₁₋₂alkyl, fluoroC₁₋₂alkyl, C₁₋₂alkoxy, C₁₋₃alkanoyl, C₂alkanoylamino, fluoroC₁alkylsulfonyl and methylsulfonyl; and
Y is phenyl, heterocyclyl, a 5- or 6-membered heteroaromatic ring or a 8- to 11-membered bicyclic group, any of which is optionally substituted by 1, 2, 3 or 4 substituents selected from the group consisting of: halogen, cyano, C₁₋₂alkyl, haloC₁₋₂alkyl, C₁₋₂alkoxy, haloC₁₋₂alkoxy, C₁₋₂alkylenedioxy, C₂₋₃alkanoyl, C₂alkanoylamino, methylsulfonyl, haloC₁alkylsulfonyl, methylsulfonyloxy, methylaminosulfonyl, methylsulfonylamino and methylaminocarbonyl.

13. A compound as claimed in claim 1, which is:
7-(5-Methyl-3-isoxazolyl)-3-(2-{[4-methyl-1,3-oxazol-5-yl)-4H-1,2,4-triazol-3-yl]thio}ethyl)-2,3,4,5-tetrahydro-1H-3-benzazepine
7-(5-Methyl-3-isoxazolyl)-3-(2-{[4-methyl-5-(tetrahydro-2H-pyran-4-yl)-4H-1,2,4-triazol-3-yl]thio}ethyl)-2,3,4,5-tetrahydro-1H-3-benzazepine
7-(5-Methyl-3-isoxazolyl)-3-(2-{[4-methyl-5-(2-methyl-5-quinolinyl)-4H-1,2,4-triazol-3-yl]thio}ethyl)-2,3,4,5-tetrahydro-1H-3-benzazepine
7-(5-Methyl-3-isoxazolyl)-3-(2-{[4-methyl-5-(2-methyl-6-quinolinyl)-4H-1,2,4-triazol-3-yl]thio}ethyl)-2,3,4,5-tetrahydro-1H-3-benzazepine
7-(1,3-Dimethyl-1H-pyrazol-5-yl)-3-(2-{[4-methyl-5-(2-methyl-5-quinolinyl)-4H-1,2,4-triazol-3-yl]thio}ethyl)-2,3,4,5-tetrahydro-1H-3-benzazepine
7-(1,3-Dimethyl-1H-pyrazol-5-yl)-3-(2-{[4-methyl-5-(5-methyl-2-pyrazinyl)-4H-1,2,4-triazol-3-yl]thio}ethyl)-2,3,4,5-tetrahydro-1H-3-benzazepine
3-(2-{[5-(3,4-Difluorophenyl)-4-methyl-4H-1,2,4-triazol-3-yl]thio}ethyl)-7-(1,3-dimethyl-1H-pyrazol-5-yl)-2,3,4,5-tetrahydro-1H-3-benzazepine
7-(5-Methyl-3-isoxazolyl)-3-(2-{[4-methyl-5-(2-methyl-3-pyridinyl)-4H-1,2,4-triazol-3-yl]thio}ethyl)-2,3,4,5-tetrahydro-1H-3-benzazepine formate
7-(5-Methyl-3-isoxazolyl)-3-(2-{[4-methyl-5-(4-pyridazinyl)-4H-1,2,4-triazol-3-yl]thio}ethyl)-2,3,4,5-tetrahydro-1H-3-benzazepine formate
7-(5-Methyl-3-isoxazolyl)-3-[2-({4-methyl-5-[2-methyl-6-(trifluoromethyl)-3-pyridinyl]-4H-1,2,4-triazol-3-yl}thio)ethyl]-2,3,4,5-tetrahydro-1H-3-benzazepine formate
3-(2-{[5-(1,5-Dimethyl-1H-pyrazol-4-yl)-4-methyl-4H-1,2,4-triazol-3-yl]thio}ethyl)-7-(5-methyl-3-isoxazolyl)-2,3,4,5-tetrahydro-1H-3-benzazepine formate
3-(2-{[5-(5-Chloro-1-methyl-1H-pyrazol-4-yl)-4-methyl-4H-1,2,4-triazol-3-yl]thio}ethyl)-7-(5-methyl-3-isoxazolyl)-2,3,4,5-tetrahydro-1H-3-benzazepine formate
7-(5-Methyl-3-isoxazolyl)-3-[2-({4-methyl-5-[4-(trifluoromethyl)phenyl]-4H-1,2,4-triazol-3-yl}thio)ethyl]-2,3,4,5-tetrahydro-1H-3-benzazepine formate
3-(2-{[5-(3,4-Difluorophenyl)-4-methyl-4H-1,2,4-triazol-3-yl]thio}ethyl)-7-(5-methyl-3-isoxazolyl)-2,3,4,5-tetrahydro-1H-3-benzazepine formate
7-(5-Methyl-3-isoxazolyl)-3-(2-{[4-methyl-5-(5-methyl-2-pyrazinyl)-4H-1,2,4-triazol-3-yl]thio}ethyl)-2,3,4,5-tetrahydro-1H-3-benzazepine formate
3-(2-{[1-(1-Methylethyl)-5-(methylsulfonyl)-1H-benzimidazol-2-yl]thio}ethyl)-7-(5-methyl-3-isoxazolyl)-2,3,4,5-tetrahydro-1H-3-benzazepine formate or a pharmaceutically acceptable salt thereof.

14. A process for preparing a compound as defined in claim 1, which process comprises:
(a) reacting a compound of formula (II): 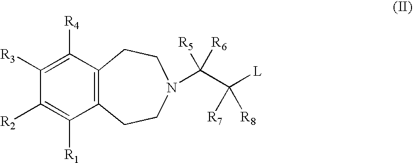
wherein R₁ to R₈ are as defined for formula (I) and L is a leaving group; with a compound of formula (III): 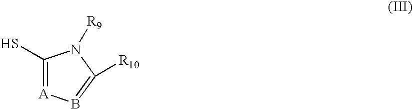
wherein A, B, R₉ and R₁₀ are as defined for formula (I); or 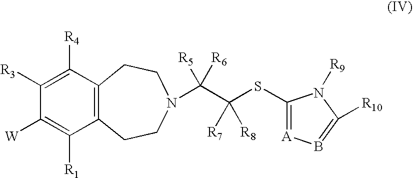
(b) for a compound of formula (I) wherein R₂ is aryl, reacting a compound of formula (IV):
wherein R₁, R₃ to R₁₀, A and B are as defined for formula (I) and W is halogen or a trifluoromethylsulfonyloxy group, or W is a group M selected from a boron derivative (e.g. a boronic acid function B(OH)₂) or a metal function such as trialkylstannyl (e.g. SnBu₃), zinc halide or magnesium halide; with a compound aryl-W¹, wherein aryl is as defined for formula (I), W¹ is halogen or a trifluoromethylsulfonyloxy group when W is a group M or W¹ is a group M as defined above when W is halogen or a trifluoromethylsulfonyloxy group; or
(c) for a compound of formula (I) wherein R₂ is aryloxy or arylthio, reacting a compound of formula (V): 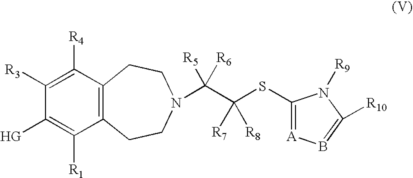
wherein G is oxygen or sulfur, and R₁, R₃ to R₁₀, A and Bare as defined for formula (I); with a reagent serving to introduce the aryl group;
and optionally thereafter for any of the steps (a), (b) or (c):
removing any protecting group(s); and/or
forming a salt; and/or
converting one compound of formula (I) to a different compound of formula (I).

15. A method of treating a condition for which modulation of dopamine D₃ receptors is beneficial, which comprises administering to a mammal (e.g. human) in need thereof an effective amount of a compound of a compound of claim 1.

16. A method as claimed in claim 15, wherein the condition is substance abuse and/or drug dependency.

17. A method as claimed in claim 16, wherein the condition is craving for abused substance and/or relapse to drug seeking and drug taking behaviour.

18-20. (canceled)

21. A compound as claimed in claim 1 for use in therapy.

22. A compound as claimed in claim 1 for use in the treatment of a condition in a mammal for which modulation of dopamine D3 receptors is beneficial.

23. A compound as claimed in claim 1 for use in the treatment of substance abuse and/or drug dependency.

24. (canceled)

25. A pharmaceutical composition comprising a compound as claimed in claim 1 and a pharmaceutically acceptable carrier.
